(19) United States
(12) Patent Application Publication
(10) Pub. No.: US 2022/0000364 A1
(43) Pub. Date: Jan. 6, 2022

(54) WIDE FIELD OF VIEW EYE IMAGING AND/OR MEASURING APPARATUS

(71) Applicant: Tesseract Health, Inc., Guilford, CT (US)

(72) Inventor: Tyler S. Ralston, Clinton, CT (US)

(21) Appl. No.: 17/365,586

(22) Filed: Jul. 1, 2021

Related U.S. Application Data
(60) Provisional application No. 63/155,866, filed on Mar. 3, 2021.
(60) Provisional application No. 63/127,962, filed on Dec. 18, 2020.
(60) Provisional application No. 63/047,536, filed on Jul. 2, 2020.

(51) Int. Cl. A61B 3/14; A61B 3/00; A61B 3/12

(52) U.S. Cl. CPC A61B 3/14; A61B 3/0041; A61B 3/0075; A61B 3/12; A61B 3/0091

(57) ABSTRACT

The present disclosure provides improved techniques for imaging and/or measuring a subject's eye. Various aspects of the present disclosure relate to an imaging and/or measuring apparatus. Some aspects of the present disclosure relate to an imaging and/or measuring apparatus comprising a lens at least capable of providing a 30 degree field of view of a subject's eye. Some aspects of the present disclosure relate to an imaging and/or measuring apparatus comprising a plurality of imaging and/or measuring optical components configured to: when a first objective lens is positioned between the plurality of imaging and/or measuring optical components and a subject's eye, transmit and/or receive light having a first set of optical characteristics; and when a second objective lens is positioned between the plurality of imaging and/or measuring optical components and the subject's eye, transmit and/or receive light a second set of optical characteristics.

### CROSS-REFERENCE TO RELATED APPLICATIONS

This application claims the benefit under 35 U.S.C. § 119(e) of U.S. Provisional Patent Application Ser. No. 63/047,536, filed on Jul. 2, 2020, under Attorney Docket No. T0753.70022US00, and entitled “NOVEL FUNDUS IMAGER”; U.S. Provisional Patent Application Ser. No. 63/127,962, filed on Dec. 18, 2020, under Attorney Docket No. T0753.70021US00, and entitled “DEVICE-ASSISTED EYE IMAGING AND/OR MEASUREMENT”; and U.S. Provisional Patent Application Ser. No. 63/155,866, filed on Mar. 3, 2021, under Attorney Docket No. T0753.70022US01, and entitled “PORTABLE EYE IMAGING AND/OR MEASURING APPARATUS”, each application of which is hereby incorporated herein by reference in its entirety.

### FIELD OF THE DISCLOSURE

The present disclosure relates to techniques for imaging and/or measuring a subject's eye, including the subject's retina fundus.

### BACKGROUND

Techniques for imaging and/or measuring a subject's eye would benefit from improvement.

### SUMMARY OF THE DISCLOSURE

An imaging and/or measuring apparatus comprising a lens at least capable of providing a 30 degree field of view of a subject's eye.

An imaging and/or measuring apparatus comprising a plurality of imaging and/or measuring optical components configured to: when a first objective lens is positioned between the plurality of imaging and/or measuring optical components and a subject's eye, transmit and/or receive light having a first field of view; and when a second objective lens is positioned between the plurality of imaging and/or measuring optical components and the subject's eye, transmit and/or receive light having a second field of view.

A method of imaging and/or measuring a subject's eye, the method comprising: receiving light from the subject's eye; transmitting the received light to a detector using a plurality of imaging and/or measuring optical components, the plurality of imaging and/or measuring optical components capable of providing a 30 degree field of view; and detecting an image and/or measurement.

An imaging and/or measuring apparatus configured to capture an image and/or measurement of a subject's eye, the imaging and/or measuring apparatus comprising: a plurality of illumination optical components comprising a spatial filter; and an objective lens configured to transmit and/or receive light with a field of view of the subject's eye.

A method of imaging and/or measuring a subject's eye, the method comprising: generating an annular illumination profile; attenuating a portion of the illumination profile using a spatial filter; and transmitting the attenuated illumination profile to a subject's retina fundus using an objective lens.

The foregoing summary is not intended to be limiting. Moreover, various aspects of the present disclosure may be implemented alone or in combination with other aspects.

### BRIEF DESCRIPTION OF FIGURES

FIG. 1A is a top perspective view of an exemplary imaging and/or measuring apparatus, according to some embodiments.

FIG. 1B is an exploded view of the imaging and/or measuring apparatus of FIG. 1A, according to some embodiments.

FIG. 1C is side view of a subject operating the imaging and/or measuring apparatus of FIG. 1A seated in a stand, according to some embodiments.

FIG. 2 is a top perspective view of an exemplary imaging and/or measuring apparatus having multiple housing portion removed to show white light imaging, fluorescence, optical coherence tomography (OCT), and infrared (IR) imaging and/or measuring components, according to some embodiments.

FIG. 3A is a diagram of the field of view of a first configuration of optical components of the imaging and/or measuring apparatus, according to some embodiments.

FIG. 3B is a diagram of the field of view of a second configuration of optical components of the imaging and/or measuring apparatus, according to some embodiments.

FIG. 3C is a diagram of the field of view of a third configuration of optical components of the imaging and/or measuring apparatus, according to some embodiments.

FIG. 4A is a diagram of a spatial filter configuration that attenuates a portion of light emitted from a light source, according to some embodiments.

FIG. 4B is a diagram of another spatial filter configuration that attenuates a portion of light emitted from a light source, according to some embodiments.

FIG. 5A is a diagram of exemplary source, fixation, and detection components that may be included in the imaging and/or measuring apparatus, according to some embodiments.

FIG. 5B is a diagram of exemplary white light detection, fixation, source, and florescence detection components that may be included in the imaging and/or measuring apparatus of FIG. 2, according to some embodiments.

FIG. 6 is a top view of a portion of the imaging and/or measuring apparatus of FIG. 2 showing the white light and fluorescence detection components of the imaging and/or measuring apparatus, according to some embodiments.

FIG. 7 is a schematic view of the source, fixation, and detection components of FIG. 6, according to some embodiments.

FIG. 8A is a front view of light source components that may be included in white light and fluorescence imaging components, according to some embodiments.

FIG. 8B is a front view of alternative light source components that may be included in white light and fluorescence imaging components, according to some embodiments.

FIG. 8C is a front view of a plate of white light and fluorescence imaging components, according to some embodiments.

FIG. 8D is a front view of a plate with an obscuration of white light and fluorescence imaging components, according to some embodiments.

FIG. 8E is a front view of illumination mirror of white light and fluorescence imaging components, according to some embodiments.

FIG. 9A illustrates sample and detect components of an imaging and/or measuring apparatus, in accordance with some embodiments.

FIG. 9B is a diagram of another configuration of detection components, according to some embodiments.

FIG. 10A is a schematic view of a configuration of the illumination, fixation, and detection components, according to some embodiments.

FIG. 10B is a schematic view of another configuration of the illumination, fixation, and detection components, according to some embodiments.

FIG. 11A is a diagram of an objective lens, according to some embodiments.

FIG. 11B is a diagram of another objective lens, according to some embodiments.

FIG. 12 is a diagram illustrating differences between a first configuration of detection components and a second configuration of detection components, according to some embodiments.

FIG. 13 is a diagram illustrating differences between a first configuration of illumination components and a second configuration of detections components, according to some embodiments.

FIG. 14 is a diagram illustrating differences between a first configuration of fixation components and a second configuration of fixation components, according to some embodiments.

FIG. 15A illustrates an illumination profile generated by the imaging and/or measuring device at a distance corresponding to the subject's pupil during imaging and/or measuring, in accordance with some embodiments.

FIG. 15B illustrates an illumination profile 508 generated by the imaging and/or measuring device at a distance corresponding to the subject's pupil during imaging and/or measuring, in accordance with some embodiments.

FIG. 15C is a plot illustrating exemplary illumination profiles generated by the imaging and/or measuring device at a subject's pupil, according to some embodiments.

FIG. 15D is a plot illustrating exemplary illumination profiles generated by the imaging and/or measuring device at a subject's pupil, according to some embodiments.

FIG. 16A is an exemplary illumination profile generated by the imaging and/or measuring device at the holed mirror in FIG. 5A, according to some embodiments.

FIG. 16B is another exemplary illumination profile generated by the imaging and/or measuring device at the holed mirror in FIG. 5A, according to some embodiments.

FIG. 17A is an exemplary illumination profile generated by the imaging and/or measuring device at a subject's retina, according to some embodiments.

FIG. 17B is an exemplary illumination profile generated by the imaging and/or measuring device at a subject's retina, according to some embodiments.

FIG. 18 is a flowchart of a method of detecting an image and/or measurement of a subject's eye, according to some embodiments.

FIG. 19 is a flowchart of a method of illuminating a subject's eye, according to some embodiments.

### DETAILED DESCRIPTION

I. Introduction to Wide-Angle Field of View, Spatial Filtering, and Interchangeable Optical Components for Imaging and/or Measuring Techniques

The present disclosure provides techniques for improving the performance and versatility of eye imaging and/or measuring components. Some techniques described herein provide optical components configured to provide a wide-angle field of view. In some applications, wide-angle field of view optical components may provide advantages in resolving features from a subject's eye. Some techniques described herein provide interchangeable optical components for providing different imaging and/or measuring properties. The relevant features of interest for determining different health conditions, based on imaging and/or measuring a subject's eye, may vary between different health conditions. In determining some health conditions, a wider field of view of a subject's tissue may provide advantages in resolving relevant features. For example, a wider field of view may provide a larger portion of the tissue in the image and/or measurement for analysis. However, in determining other health conditions, a narrower field of view of a subject's tissue may provide advantages in resolving relevant features. For example, a feature of the tissue depicted in a narrower field of view may appear larger than in a wider field of view. Accordingly, interchangeable optical components which are configured to provide different fields of view, when configured with an eye imaging and/or measuring apparatus may improve the versatility of the apparatus. Some techniques described herein provide spatial filtering techniques to reduce the amount of scattered light reaching a detector and decreasing the imaging and/or measurement quality.

The inventors have recognized and appreciated that a person's eyes provide a window into the body that may be used to not only determine whether the person has an ocular disease, but to determine the general health of the person. The retina fundus, in particular, can provide valuable information via imaging for use in various health determinations. However, existing eye imaging systems only provide superficial information about the subject's eye and cannot provide sufficient information to diagnose certain diseases.

The inventors have further recognized and appreciated that making the device compact and affordable would have the greatest impact on global health. Accordingly, some embodiments are directed to an apparatus that includes multiple modes of imaging the retina fundus within a housing. For example, a white light imaging system, an illumination system, and a fixation system may be housed in a binocular housing that may optionally include at least one of a fluorescence detection system, an optical coherence tomography system, or infrared (IR) imaging system. Additionally, for some applications, making the device easy to use, accurate, high resolution, and capable of cloud connectivity and/or other remote communications would further impact global health.

The inventors have appreciated that an apparatus with a wide-angle field of view may be advantageous in some embodiments for providing a wider field of view of the tissue for analysis and/or making health determinations. However, the inventors have also appreciated that obtaining a wide field of view using a portable imaging and or measuring apparatus presents further challenges. One technique that can be used to provide wide-angle imaging (and/or measuring) is to use an optical scanner that uses movable mirrors to provide a wide-angle field of view by rastering a narrow field of view of the illumination and/or the detection light over a wider area and reconstructing a wide-angle field of view. The inventors have appreciated that optical components that are designed to move during use (e.g., an optical scanner) may be more susceptible to misalignment than stationary optical components (e.g., a mirror or lens), making them unsuitable for some applications.

The inventors have appreciated that developing an imaging and/or measuring apparatus configured to provide a wide-angle field of view of a subject's eye presents additional challenges, as, for example, the size of a subject's pupil may restrict the field of view provided by an imaging and/or measuring apparatus. One way to increase the field of view of an imaging and/or measuring apparatus is to dilate the pupil. The dilated pupil enables more light to enter the eye. However, pupil dilation requires a mydriatic agent (e.g., a drug that induces pupil dilation) and may result in blurred vision and increased sensitivity to light until the pupil dilating effects wear off, which usually takes several hours. As such, eye dilation can require a substantial amount of a subject's time. For some applications, the duration of pupil dilating effects may make dilation-based imaging and/or measuring the subject's fundus less convenient and much more difficult to do on a regular basis.

Recognizing the above challenges, the inventors have developed an imaging and/or measuring apparatus that includes an objective lens at least capable of providing a 30 degree field of view of a subject's retina fundus. For example, in some embodiments, the apparatus includes an objective lens, illumination optical components that are configured to transmit light to the objective lens to illuminate a portion of the subject's eye, and fixation optical components configured to transmit light to the objective lens to provide a visual indicator to the subject's eye. The visual indicator may provide visual feedback to indicate the position of the subject's eye relative to a wide-angle field of view of the apparatus. The light transmitted by the illumination optical components provides illumination of the subject's retina fundus. In some embodiments, all of the optics of the apparatus are capable of providing a field of view of at least 30 degrees. In some embodiments, at least some of the optics of the apparatus, while capable of providing a 30 degree field of view, may be configured to provide a narrower field of view (i.e., less than 30 degrees), as the field of view may be limited by one or more angle limiting optical components. For example, the angle-limiting optical components can include an aperture, a lens, and/or a detector that are configured to provide a narrower field of view than the remaining optics of the apparatus, which can limit the extent to which the optics illuminate the angle-limiting components.

It should be appreciated that optics capable of providing at least a 30 degree field of view may also be capable of providing a wider field of view (i.e., greater than 30 degrees). For example, a lens capable of providing a 60 degree field of view may be capable of providing any field of view angle from 0 degrees to 60 degrees. In some embodiments, a lens capable of providing a 60 degree field of view could be used to provide a 30 degree field of view to a detector, where the detector is configured to only receive a 30 degree field of view from the lens (e.g., as limited by an optical component capable of providing, at most, a 30 degree field of view). Other fields of view are also possible, as aspects of the technology described herein are not limited in this respect. Furthermore, the phrase “capable of providing an X degree field of view,” as used herein, should be understood to mean “capable of providing at least an X degree field of view.”

The inventors have further recognized that, for some applications, it is desirable to use different fields of view when capturing images and/or measurements of a subject's retina fundus. For example, it may be advantageous to use a wide field of view to capture an image and/or measurement of a first a portion of the retina fundus. In this example, it may also be advantageous to capture an image and/or measurement of a second portion of the fundus using a narrower field of view, such as when the second portion is contained within the first portion (e.g., as a follow up to identifying a potential problem in the first portion). However, existing apparatuses are not capable of supporting different fields of view for imaging and/or measuring.

To address this problem, the inventors developed imaging and/or measuring apparatuses that can be configured to transmit and/or receive light having different fields of view using different objective lenses. For example, in some embodiments, an imaging and/or measuring apparatus can include optical components configured to transmit and/or receive light having a first field of view when a first objective lens is positioned between the optical components and a subject's eye. For example, the first objective lens may be capable of providing up to a 30 degree field of view, and the optical components can thereby be limited to providing up to a 30 degree field of view. In some embodiments, the optical components can be configured to transmit and/or receive light having a second field of view when a second objective lens is positioned between the optical components and the subject's eye. For example, the second objective lens may be capable of providing up to a 45 degree field of view, and the optical components can thereby be limited to providing up to a 45 degree field of view. By including optical components that can be configured to support different fields of view with different objective lenses, an imaging and/or measuring apparatus can be flexibly configured to capture images and/or measurements with different fields of view, which can be useful for providing medical quality images and/or measurements with varying magnifications using a compact, portable apparatus.

The inventors have appreciated that illumination of the fundus presents challenges. Illumination of the retina fundus is difficult because the eye is a multilayered organ, and the fundus is located on the cornea at the back of the eye. Accordingly, reflections of the illumination light from the other layers of the eye (e.g., cornea and/or lens) may result in scattered light at the detector, decreasing the signal to noise ratio and by extension decreasing the sensitivity of the detection. Additionally, the surfaces of optical components configured between the eye and the detector may also scatter unwanted reflections into the detector. Polarization techniques have been used to reduce the scattering of light to the detector, thereby increasing the contrast of the resulting image and/or measurement. However, using polarization optical components to create polarized illumination, and/or to filter out light scattered with a particular polarization, results in losses to the optical power and may block certain signal components useful to diagnosing a subject, decreasing the illumination and/or signal intensity and making polarization techniques unsuitable for some applications. The lost optical power corresponds to wasted electrical power and reduces the efficiency of a portable, battery powered apparatus. Additionally, polarization optical components are more expensive than their non-polarizing counterparts. The inventors have recognized that illumination optical components that illuminate the fundus and reduce scattered light to the detector without using polarization components are desirable to increase power efficiency and decrease cost of a portable apparatus.

The inventors have appreciated that supplying a portable apparatus for imaging and/or measuring a subject's retina fundus may compound these challenges. In a portable apparatus, longer battery life and smaller/lighter batteries can be supported by energy efficient systems, increasing the commercial value of the product. Light generation can be energy intensive. To image and/or measure the eye, illumination and/or excitation light is transmitted into the eye and then scattered (e.g., reflected and/or emitted) light is collected and transmitted to detection optical components.

To address the problems above, the inventors have developed imaging and/or measuring apparatuses that include illumination components to reduce scattered light to the detector using a spatial filter in the illumination optical path. For example, in some embodiments, an imaging and/or measuring apparatus configured to capture an image and/or measurement of a subject's retina fundus includes illumination optical components including a spatial filter, and an objective lens configured to transmit and/or receive light with a field of view of the subject's eye (e.g., receive light reflected by or emitted from the subject's eye). By including the spatial filter with the illumination components, light that would be scattered to the detector from the surfaces of optical components, as well as light that would be scattered to the detector from the surface of the eye can be selectively blocked, without attenuating all the illumination light. Therefore, more of the generated light may be used for imaging, providing for more energy efficient illumination.

The aspects and embodiments described above, as well as the additional aspects and embodiments, that are described further below, may be used individually, all together, or in any combination of two or more, as the technology described herein is not limited in this respect.

II. Exemplary Imaging and/or Measuring Apparatus

Techniques described herein such as techniques including spatial filters, wide angle fields of view, and interchangeable objective lenses can be employed in various imaging and/or measuring apparatuses, an example of one such exemplary apparatus is described further herein.

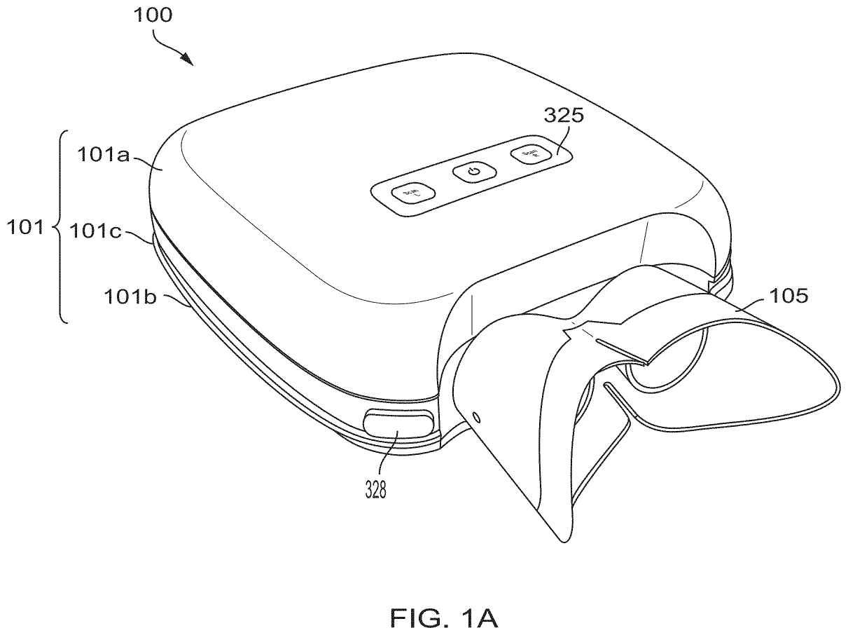

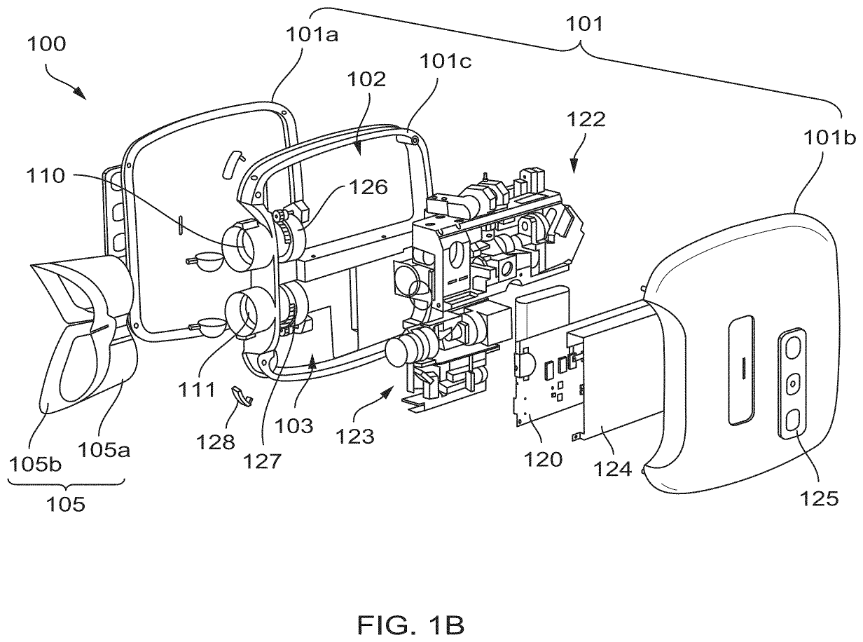

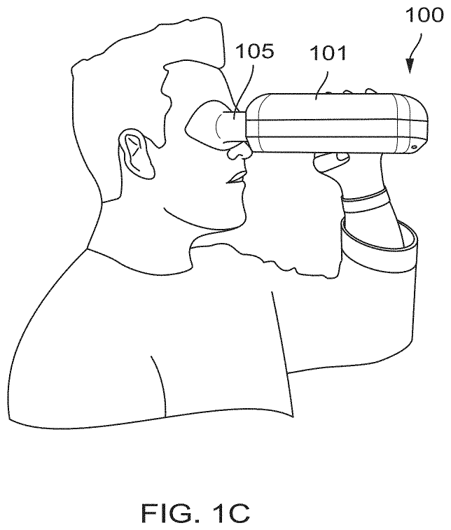

FIGS. 1A-1C illustrate an exemplary embodiment of an imaging (and/or measuring apparatus) 100, according to some embodiments. As shown in FIG. 1A, imaging apparatus 100 has a housing 101, including multiple housing portions 101a, 101b, and 101c. Housing portion 101a has a control plane 125 including multiple buttons for turning imaging apparatus 100 on or off, and for initiating scan sequences. FIG. 1B is an exploded view of imaging apparatus 100 illustrating components disposed within housing 101, such as imaging (and/or measuring) devices 122 and 123 and electronics 120. Imaging devices 122 and 123 may include multiple detection modes, including one or more of: white light imaging components, fluorescence imaging components, infrared (IR) imaging components, and/or OCT imaging components, in accordance with various embodiments. In one example, imaging device 122 may include OCT imaging components and/or IR imaging components, and imaging device 123 may include white light imaging components and/or fluorescence imaging components. In some embodiments, imaging device 122 and/or 123 may include fixation components configured to display a visible fixation object to the subject. Imaging apparatus 100 further includes front housing portion 105 configured to receive a subject's eyes for imaging, as illustrated, for example, in FIG. 1C.

As shown in FIGS. 1A-1C, housing portions 101a and 101b may substantially enclose imaging apparatus 100, such as by having all or most of the components of imaging apparatus 100 disposed between housing portions 101a and 101b. Housing portion 101c may have multiple housing portions therein, such as housing portions 102 and 103 for accommodating imaging devices 122 and 123. For example, in some embodiments, the housing portions 102 and 103 may be configured to hold imaging devices 122 and 123 in place. Housing portion 101c further includes a pair of lens portions in which lenses 110 and 111 are disposed. Housing portions 102 and 103 and the lens portions may be configured to hold imaging devices 122 and 123 in alignment with lens 110 and 111. Housing portions 102 and 103 may accommodate focusing parts 126 and 127 for adjusting the foci of lenses 110 and 111. Some embodiments may further include securing tabs 128. By adjusting (e.g., pressing, pulling, pushing, etc.) securing tabs 128, housing portions 101a, 101b, and/or 101c may be decoupled form one another, such as for access to components of imaging apparatus 100 for maintenance, adjustment, and/or repair purposes.

As shown in FIG. 1B, electronics 120 of imaging apparatus 100 may be configured to perform imaging, measuring, and/or associated processing. In some embodiments, electronics 120 may include one or more processors, such as for analyzing data captured using the imaging devices. In some embodiments, electronics 120 may include wired and/or wireless means of electrically communicating to other devices and/or computers, such as a mobile phone, desktop, laptop, or tablet computer, and/or smart watch. For example, electronics 120 of imaging apparatus 100 may be configured for establishing a wired and/or wireless connection to such devices, such as by USB and/or suitable wireless network. In some embodiments, housing 101 may include one or more openings to accommodate one or more electrical (e.g., USB) cables. In some embodiments, housing 101 may have one or more antennas disposed thereon for transmitting and/or receiving wireless signals to or from such devices. In some embodiments, imaging devices 122 and/or 123 may be configured for interfacing with the electrical cables and/or antennas. In some embodiments, electronics 120 may be configured to process captured image data based on instructions received from such communicatively coupled devices or computers. In some embodiments, imaging apparatus 100 may initiate an image capture sequence based on instructions received from devices and/or computers communicatively coupled to imaging apparatus 100. In some embodiments, devices and/or computers communicatively coupled to imaging apparatus 100 may process image data captured by imaging apparatus 100. In some embodiments, imaging apparatus 100 may include a battery configured to provide power for operating electronics 120 and imaging devices 122 and 123. For example, imaging apparatus 100 may be configured to capture and/or analyze captured images using power supplied from the battery, such that imaging apparatus 100 may be portable and configured to capture and process medical grade images using techniques, and appropriate fixation components and illumination components, such as white light imaging, fluorescence imaging, optical coherence tomography, infrared imaging and/or other techniques, as described further herein.

Control panel 125 may be electrically coupled to electronics 120. For example, the scan buttons of control panel 125 may be configured to communicate an image capture and/or scan command to electronics 120 to initiate a scan using imaging device 122 and/or 123. As another example, the power button of control panel 125 may be configured to communicate a power on or power off command to electronics 120. As illustrated in FIG. 1B, imaging apparatus 100 may further include electromagnetic shielding 124 configured to isolate electronics 120 from sources of electromagnetic interference (EMI) in the surrounding environment of imaging apparatus 100. Including electromagnetic shielding 124 may improve operation (e.g., noise performance) of electronics 120. In some embodiments, electromagnetic shielding 124 may be coupled to one or more processors or electronics 120 to dissipate heat generated in the one or more processors.

As shown in FIG. 1C, for example, during operation of the imaging apparatus 100, a person using the imaging apparatus 100 may place the front housing section 105 against the person's face such that the person's eyes are aligned with the lens portions of imaging apparatus 100. In some embodiments, the imaging apparatus 100 may include a gripping member (not shown) coupled to the housing 101 and configured for gripping by a person's hand. In some embodiments, the gripping member may be formed using a soft plastic material and may be ergonomically shaped to accommodate the person's fingers. For instance, the person may grasp the gripping member with both hands and place the front housing section 105 against the person's face such that the person's eyes are in alignment with the lens portions.

Additionally, or alternatively, imaging (and/or measuring) apparatuses described herein may be configured for mounting to a stand, and/or a mount to be positions on a part of a subject, in accordance with some embodiments.

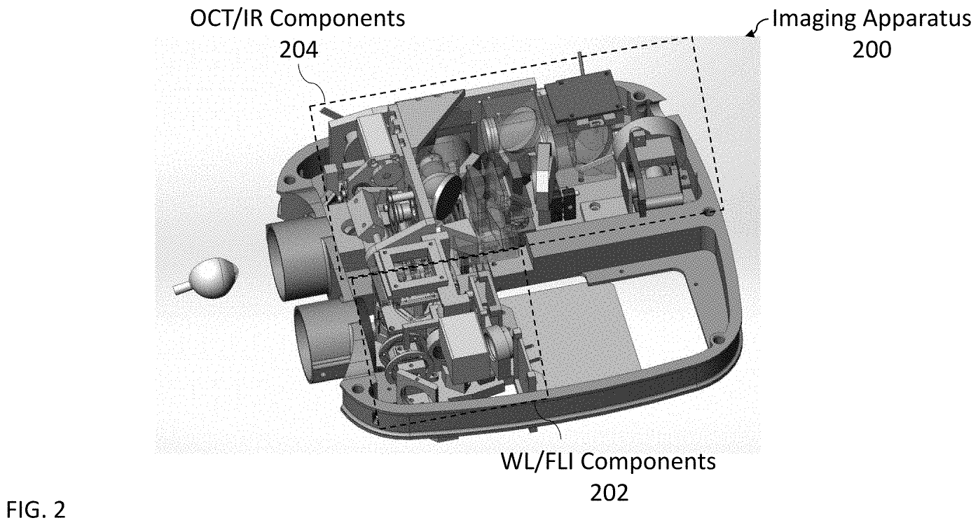

FIG. 2 is a top perspective view of an exemplary imaging and/or measuring apparatus 200 having multiple housing portions removed to show white light and/or fluorescence imaging and/or measuring components 202 and OCT and/or IR imaging and/or measuring components 204, according to some embodiments. As shown in FIG. 2, a first side of imaging and/or measuring apparatus 200 has white light and/or fluorescence components 202 and a second side of imaging and/or measuring apparatus 200 has OCT and/or IR components 204.

In some embodiments, OCT components of OCT and/or IR components 204 may be configured to illuminate a subject's eye with light from a light source (e.g., a super-luminescent diode) and compare light reflected from the subject's eye with light reflected from a reference surface to capture an image (e.g., one or more depth scans) of the subject's eye. In some embodiments, IR components of OCT and/or IR components 204 may be configured to illuminate a subject's eye with IR light from an IR light source and receive IR light from the subject's eye to capture an image of the subject's eye.

As discussed above, the imaging and/or measuring apparatus may be configured with multiple detection modes. Described herein are exemplary configurations of white light and fluorescence imaging and/or measuring components. Although some exemplary configurations, illustrated herein, include each of white light and fluorescence imaging and/or measuring components, it should be appreciated that white light and/or fluorescence imaging and/or measuring components described herein may be included alone or in combination with one another and/or with other modes of imaging and/or measuring devices.

III. Wide-Angle Field of View, Spatial Filtering, and Interchangeable Optical Component Techniques

As discussed above, the field of view of an imaging and/or measuring apparatus is related to the area from which light is acquired for an image and/or measurement. A wider field of view receives light from a larger area. By contrast, a narrower field of view receives light from a smaller area. For example, images acquired of a subject's retina fundus using a wider field of view will show a larger portion of the eye than images acquired of a subject's retina fundus using a narrower field of view. If the acquired images are the same size (e.g., same dimensions in pixels) then features of the eye will appear different sizes in the different fields of view. Accordingly, for some applications, a wide field of view may provide advantages for medical diagnostics by providing a view of a larger area of the eye in a single image and/or measurement. Furthermore, for some applications, a narrower field of view may provide advantages for medical diagnostics by providing a view of a smaller portion of the eye but with the features appearing larger in the image and/or measurement. The field of view for an imaging and/or measuring apparatus depends on the configuration of the optics between the surface(s) being imaged and the detector. Therefore, depending on the configuration of the optical components, different optical components may be responsible for limiting the field of view in different configurations.

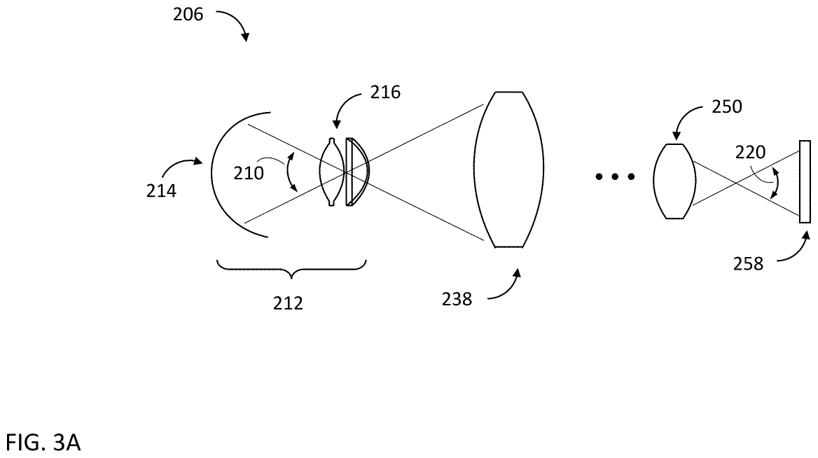

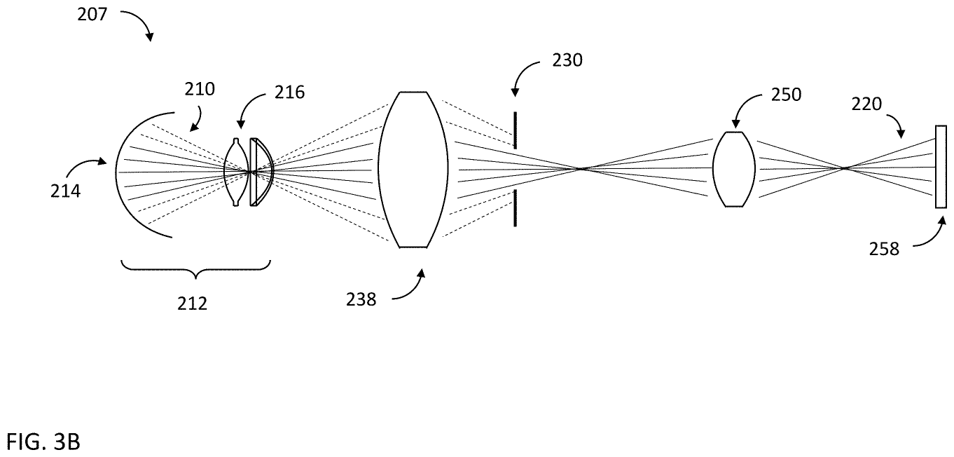

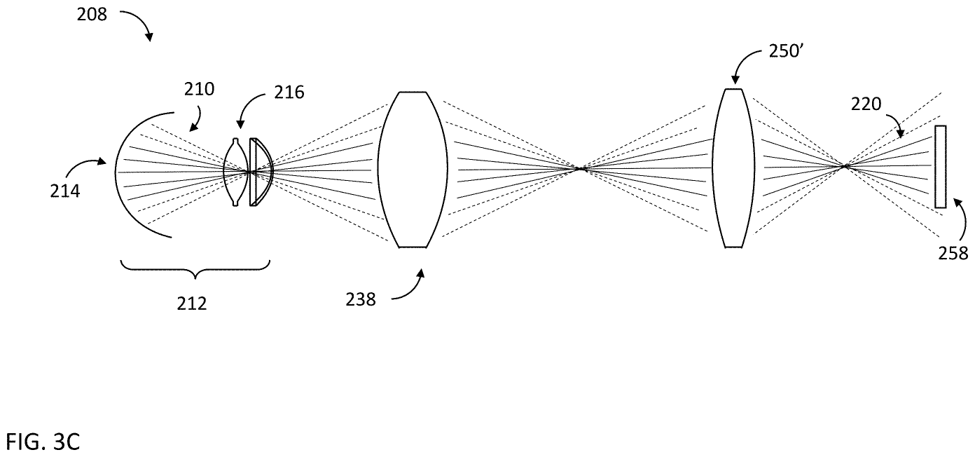

FIG. 3A is a diagram of the field of view of a first configuration 206 of optical components of the imaging and/or measuring apparatus, according to some embodiments. As shown in FIG. 3A, imaging and/or measuring component configuration 206 may include objective lens 238 configured to transmit or receive light to or from a subject's eye 212 in accordance with an angular field of view of the subject's retina fundus 214 that is determined, at least in part, on the angles that are included in the angular aperture of the lens 210 (e.g., the angles at which the lens is configured to receive light). Light received by objective 238 from subject eye 212 may be transmitted to detection optical components, including, lens 250 and detector 258. The light transmitted to detector 258 may correspond to a field of view 220. In some embodiments, additional optical components, such as those described with reference to FIGS. 2, & 4-8, may be included between objective 238 and lens 250.

The field of view, as described herein, is a measure of the angular extent of a scene (i.e., light depicting objects, surfaces, edges, and/or patterns) transmitted to a detector. Therefore, the field of view does not only depend on the lenses receiving and transmitting light, but the field of view also depends on the size of the detector and the other optics that are positioned between the scene (i.e., a subject's retina fundus) and the detector. The field of view is described according to:

$${FOV}_{i} = {tan}^{- 1} \frac{{D}_{i}}{2 f}$$

with Dᵢ being the detector size along the direction i of interest and f is the effective focal length of the system. In some embodiments, a system may have different fields of view along different dimensions of detection. For example, a detector positioned in an xy-plane may have a first field of view along the x direction and a second field of view along the y direction. In some embodiments, the first and second field of view may be different. For example, when a rectangular detector is used, the detector may be longer in a y direction such that the field of view along the y direction is wider than in the x direction. As another example, cylindrical optics may be used such that the first field of view is different from the second field of view. In some embodiments, the first and second field of view may be the same.

In some embodiments, the field of view may be further restricted by additional components that obscure portions of the transmitted light. For example, apertures, spatial filters, irises, and other components that obscure a portion of the optical path may further restrict the field of view of the apparatus. For example, an apparatus may be configured to detect images and/or measurements corresponding to a 25-35 degree field of view, a 40-50 degree field of view or a greater than 50 degree field of view. When the apparatus is configured to detect images and/or measurements corresponding to a 25-35 degree field of view, the apparatus may receive light from angles of 0 to 35 degrees or higher from a subject's eye and at least one optical component of the system may restrict the light transmitted to the detector to include light received from 0 to 25 degrees, 0 to 35 degrees, of a subset of angles there.

The inventors have appreciated that configuring optical components to transmit a wide-angle field of view of a subject's retina fundus 214 provides challenges. For example, the front portions of the eye 216 (e.g., lens, cornea, and iris) provide constraints on the light transmitted to and/or received from a subject's retina fundus. Therefore, the inventors have developed optical components configured for imaging and/or measuring a subject's retina fundus, as described herein.

FIG. 3B is a diagram of the field of view of a second configuration 207 of optical components of the imaging and/or measuring apparatus, according to some embodiments. Objective lens 238 receives light from the subject's eye 212. Light rays that are transmitted to the detector, and contribute the field of view, are shown with solid lines. By contrast, light rays that are received by the objective lens but are not transmitted to the detector 258, and do not contribute to the field of view, are shown with dashed lines. An optical component 230 may be configured to reduce the field of view by blocking a portion of the light that is transmitted by the objective lens 238 to the detector 258. As illustrated in FIG. 3B, a portion of the light rays, which are received at angles within the angular aperture of the lens 210, are blocked by an optical component 230. Therefore, optical component 230 limits the field of view 220 such that the field of view 220 includes a subset of the light rays included in the angular aperture 210. In some embodiments, optical component 230 may be configured near an intermediate image plane of the subject's eye when imaged by the optical components. For example, optical component 230 may be an aperture, as illustrated in FIG. 3B. As another example, optical component 230 may be a lens tube (not shown) associated with MV lens 250.

FIG. 3C is a diagram of the field of view of a third configuration 208 of optical components of the imaging and/or measuring apparatus, according to some embodiments. As illustrated in FIG. 3C, the field of view is limited by the size of the detector 258, light rays shown with solid lines are captured by the detector to contribute to the field of view 220. By contrast, light rays shown with dashed lines are not captured by the detector and do not contribute to the field of view. Relative to FIG. 3B, the optical components in FIG. 3C may include a MV lens 250′ that receives the same light rays that were transmitted from objective lens 238, in accordance with some embodiments. Other optical configurations are possible to modify the field of view, as aspects of the technology described herein are not limited in this respect.

The inventors have appreciated that techniques for preventing light from scattering to the detector may increase image contrast. The contrast of acquired images and/or measurements may also impact the efficiency with which features may be detected in an acquired image and/or measurement, for making a medical determination. Contrast is related to the visibility of features relative to their background (i.e., the surrounding features). For some applications, higher contrast may provide advantages for an imaging and/or measuring apparatus by enabling features to appear more clearly in the acquired images and/or measurements. However, scattered light that is received by the detector contributes to the background, decreasing contrast. For example, scattering of illumination light from internal components into the detector may decrease the contrast of acquired images and/or measurements. The inventors have recognized that light scattered from the centers of optical components and/or light that is received parallel to the central axis of the lens may contribute more to scattered light received by the detector than other light transmitted through the optical components. Accordingly, the inventors have developed optical components that incorporate spatial filters between the optical components to attenuate a portion of the light transmitted through the optical components, such that the amount of scattered light being transmitted to the detector is decreased.

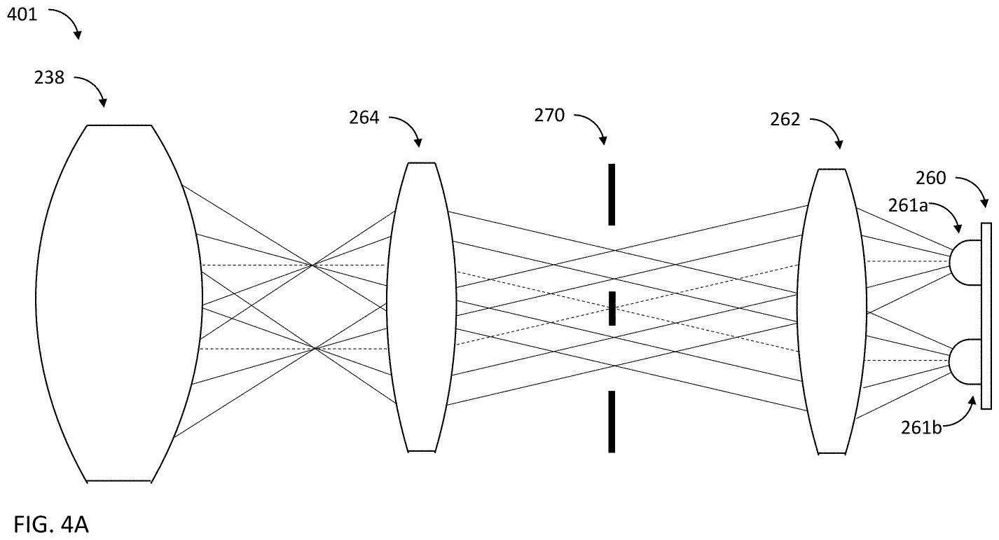

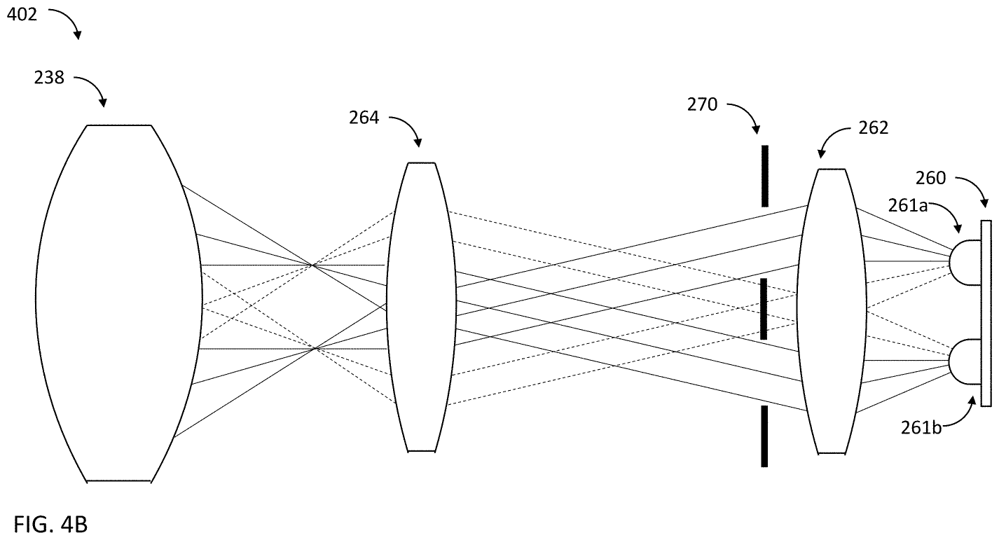

FIG. 4A is a diagram of a spatial filter configuration 401 that attenuates a portion of light emitted from a light source, in accordance with some embodiments. As shown in FIG. 4A, imaging and/or measuring components include light source 260, a first relay lens 262, a second relay lens 264, a spatial filter 270, and an objective lens 238. Light source 260 includes light emitting diodes (LEDs) 261a and 261b that emit light at multiple angles. In some embodiments, the first relay lens may be configured to collimate light received from light source 262 and the second relay lens 364 may be configured to focus the collimated light transmitted by the first relay lens 262. The objective lens 238 may be configured to receive the light transmitted from the second relay lens 264 to transmit the light, such that the transmitted light illuminates a field of view for imaging and/or measuring. A detector may be located along a different optical path that shares objective lens 238. A beam splitter, dichroic, holed mirror, or other optical component that may be configured to combine and/or separate light from shared optical paths may be configured between objective 238 and second relay lens 264 for transmitting light to the detector. In some embodiments, additional optical components (e.g., mirrors, beam splitters, dichroics, and/or additional relay lenses) may be included between the components illustrated in FIG. 4A, as aspects of the technology described herein are not limited in this respect.

In some embodiments, spatial filter 270 may be configured to attenuate a portion of the light emitted from light source 260. For example, the spatial filter 270 may be configured at an intermediate imaging plane of the optical components. In some embodiments, spatial filter 270 is configured at a conjugate optical plane where light emitted at the same angle, but different spatial positions, converges (i.e., a Fourier plane of the optical components). For example, spatial filter 270 may be configured to attenuate light emitted at an angle of 0 degrees (i.e., perpendicular to light source 260). Light emitted at an angle of 0 degrees from LED 261a and 261b is shown in FIG. 4A as a dashed line. The two rays illustrated as dashed lines being emitted from LED 261a and 261b converge near the center of the optical path, where the spatial filter 270 includes an at least partially opaque portion to attenuate the light emitted at an angle of 0 degrees. Accordingly, the light transmitted to objective lens 238 will not be received at the surface of the lens at an angle parallel to the central axis of the lens. Therefore, scattered light, such as back reflections, are less likely to be transmitted to the detector.

FIG. 4B is a diagram of another spatial filter configuration 402 that attenuates a portion of light emitted from a light source, in accordance with some embodiments. The configuration of the optical components may be similar to the configuration of FIG. 4A but with the spatial filter configured in a different position. As shown in FIG. 4B, the spatial filter may be configured to attenuate light that is transmitted to a central portion of objective lens 238. The light rays emitted from light source 260 that are attenuated by the spatial filter 270 are shown with dashed lines. Spatial filter 270 may be positioned at a conjugate optical plane that is an image plane associated with a surface of the objective lens. In some embodiments, the spatial filter may be positioned differently, or shaped differently such that the spatial filter attenuates a different portion of the illumination light, as aspects of the technology described herein are not limited in this respect.

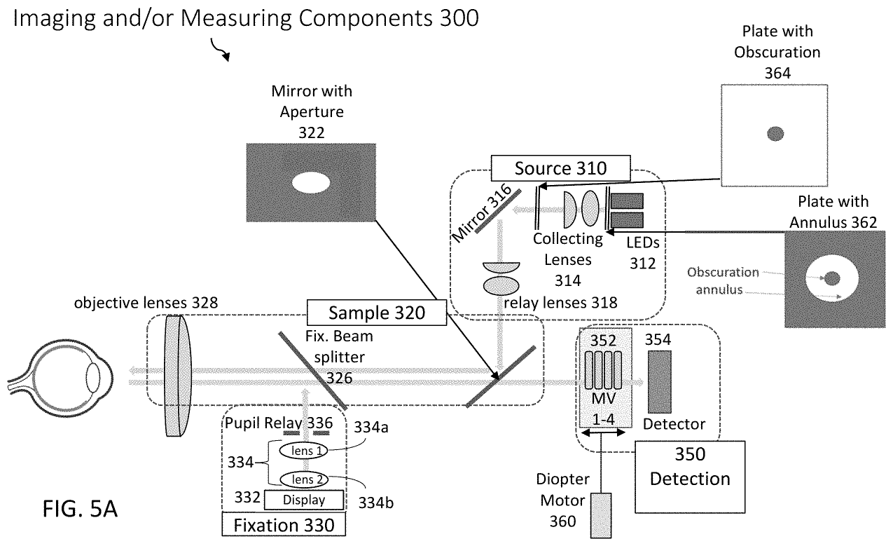

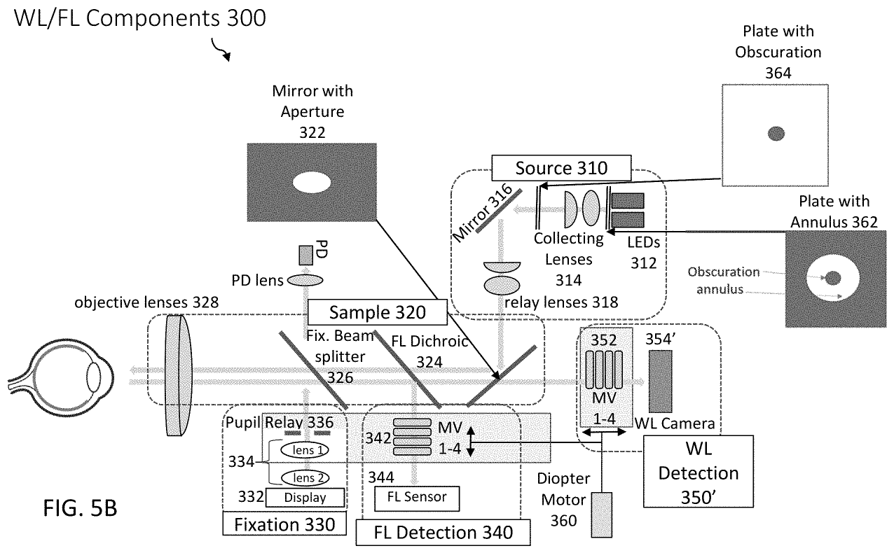

The wide-angle field of view components and spatial filter components may be used alone or in combination to implement an imaging and/or measuring apparatus. Additionally, or alternatively, the imaging and/or measuring apparatus may include an interchangeable optical component. In some embodiments, the imaging and/or measuring apparatus may include interchangeable optical components capable of providing a wide angle-field of view and spatial filtering components. FIGS. 5A-14 illustrate exemplary optical components that may be implemented in accordance with wide-angle field of view, spatial filter, and/or interchangeable optical components.

FIG. 5A is a diagram of exemplary source 310, fixation 330, and detection 350 components that may be included in the imaging and/or measuring apparatus, according to some embodiments. As shown in FIG. 5A, imaging and/or measuring source components 310 include source components 310, sample components 320, fixation components 330, and detection components 350. In some embodiments, source components 310 may be configured to provide white light and/or excitation light for illuminating and/or exciting luminescent molecules in a subject's eye via sample components 320. In some embodiments, sample components 320 may be configured to receive reflected light from the subject's eye and to provide the received light to detection components 350 to capture an image and/or measurement. In some embodiments, fixation components 330 may be configured to display to the subject's eye via sample components 330 a visible light fixation display. FIG. 5A also shows diopter motor 360, which may be configured to adjust (e.g., focus) machine vision (MV) lenses of detection components 350.

In some embodiments, sample components 320 include an objective 328 capable of providing a wide-angle field of view, as described herein. Additionally, or alternatively, objective 328 may be an interchangeable objective lens. In embodiments including an interchangeable objective lens, the source 310, fixation 330, detection 350, and sample 320 components may be configured to be used with each of the interchangeable objective lenses to produce images and/or measurements of a subject's retina fundus, as described herein.

In some embodiments, source components include a spatial filter such as plate with obscuration 364 for reducing scattered light from being transmitted to the detector 352, as described herein.

In some embodiments, the apparatus may include multiple detectors with more specialized functions (e.g., detection of specific wavelengths) and may enable acquiring different detection modes in parallel. FIG. 5B is a diagram of exemplary white light detection (350), fixation (330), source (310), and florescence detection (340) components that may be included in the imaging and/or measuring apparatus of FIG. 2, according to some embodiments. As shown in FIG. 5B, white light and fluorescence components 300 can be configured in the manner described herein for the components of FIG. 5A. In addition, the components can include fluorescence detection components 340, white light detection components 350′, and a fluorescence dichroic 324. In some embodiments, sample components 320 may be configured to receive reflected white light and/or fluorescent light from the subject's eye, provide the received fluorescent light to fluorescence detection components 340 to capture a fluorescence image, and/or provide white light to white light detection components 350′ to capture a white light image.

In some embodiments, source components 310 may be configured to generate and provide light to sample components 320 for focusing on the subject's eye such that light reflected and/or fluorescence light emitted from the subject's eye may be captured using fluorescence detection components 340 and/or white light detection components 350′. In FIG. 5B, source components 310 include LEDs 312, collecting lenses 314, mirror 316, and relay lenses 318. In some embodiments, the LEDs 312 may include white light LEDs and/or a plurality of color LEDs that combine to substantially cover the visible spectrum, thereby approximating a white light source. For example, in some embodiments, LEDs 312 may be configured to generate light having a wavelength between 400 nanometers (nm) and 700 nm. In some embodiments, LEDs 312 may combine to cover only a portion of the visible spectrum. In some embodiments, LEDs 312 may include one or more blue and/or ultraviolet (UV) lasers configured to excite autofluorescence in the subject's eye.

In some embodiments, LEDs 312 may include one or more fluorescence excitation LEDs, which may be configured to excite luminescent molecules of interest in the subject's eye. In some embodiments, LEDs 312 may be configured to generate excitation light having a wavelength between 405 460 nanometers (nm) and 450 to 500 nm, such as between 480 nm to 500 nm and/or 465 nm to 485 nm. In some embodiments, LEDs 312 may be configured to generate light having a bandwidth of 5-6 nm. In some embodiments, LEDs 312 may be configured to generate light having a bandwidth of 20-30 nm. It should be appreciated that some embodiments may include a plurality of lasers and or LEDs configured to generate light having different wavelengths.

As shown in FIG. 5B, source components 310 further include collecting lenses 314, mirror 316, and relay lenses 318. In some embodiments collecting lenses 314 may include one or more collimating lenses and relay lenses 318 may be configured to relay the collimated light to the subject's pupil. In some embodiments, mirror 316 may be configured to direct light from LEDs 312 toward sample components 320.

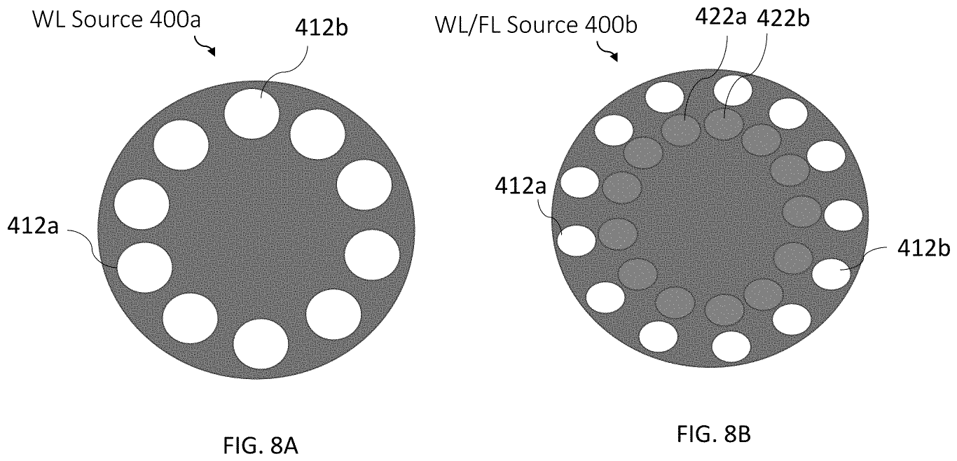

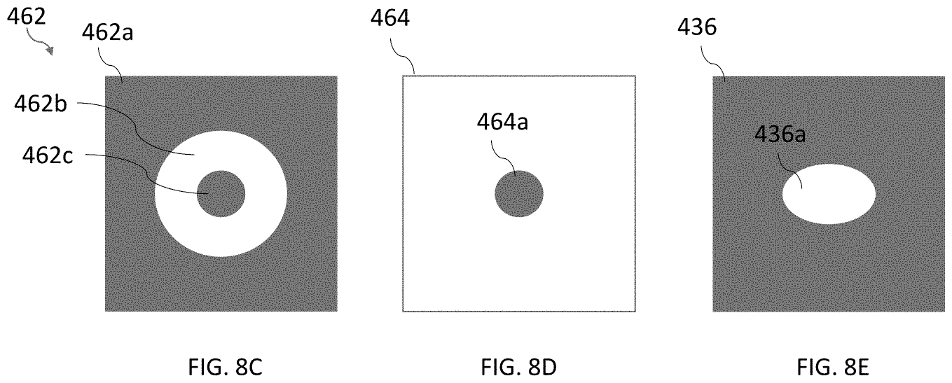

In FIG. 5B, source components 310 further include a plate 362 having an annulus, which may be positioned between LEDs 312 and collecting lenses 314. In some embodiments, plate 362 may be configured to block at least some light from LEDs 312 and transmit at least some light through the annulus. For example, in some embodiments, light transmitted through plate 362 may have a ring shape, such that the illuminated ring may be relayed to the subject's eye. Source components 310 are also shown in FIG. 5B including a plate 364 having an obscuration, which may be positioned between collecting lenses 314 and relay lenses 318. In some embodiments, plate 364 may be configured to block at least some light from reaching a portion of the subject's eye, such as the subject's cornea. The inventors have recognized that the cornea may reflect an undesirably high amount of light that can degrade the quality of images captured targeting other portions of the subject's eye. By blocking at least some light from illuminating the cornea, higher quality images may be obtained. The source components are further discussed in connection with FIGS. 8A-8F below.

In some embodiments, sample components 320 may be configured to focus light from source components 310 and fixation light from fixation components 330 on the subject's eye and provide received light (e.g., reflected and/or emitted) from the subject's eye to fluorescence detection components 340 and/or white light detection components 350′. In FIG. 5B, sample components 320 include mirror 322 having an aperture (i.e., a holed mirror), fluorescence dichroic 324, fixation beamsplitter 326, and objective lenses 328. In some embodiments, mirror 322 may be configured to receive light from source components 310 and transmit the light to the subject's eye via fluorescence dichroic 324 and fixation beamsplitter 326. In some embodiments, the aperture of mirror 322 may be configured to permit light reflected from the subject's eye to reach white light detection components 350′. For example, mirror 322 may be configured to block at least some light from the subject's eye from reaching white light detection components 350′, such as light reflected from the cornea of the subject's eye.

In some embodiments, fluorescence dichroic 324 may be configured to transmit white light and/or excitation light and reflect fluorescence light such that white light from source components 310 may reach the subject's eye and reflected white light from the subject's eye may reach white light detection components 350′, whereas fluorescence dichroic 324 may be configured to reflect fluorescent emissions from the subject's eye toward fluorescence detection components 340. For example, fluorescence dichroic 324 may be configured as a long pass filter. In some embodiments, fixation beamsplitter 326 may be configured to transmit white light, excitation light, and/or fluorescent light and reflect fixation light from fixation components 330 towards the subject's eye, such that white light and excitation light from source components 310 may reach the subject's eye and white light and/or fluorescent light received from the subject's eye may reach white light detection components 350′ and/or fluorescence detection components 340, respectively. In some embodiments, fixation beamsplitter 326 may be configured as a long pass filter. In some embodiments, fixation beamsplitter 326 may be configured to transmit light toward a photodetector (PD) and through a PD lens, where the PD is configured to determine whether the amount of light to be transmitted toward the subject's eye exceeds a safety threshold.

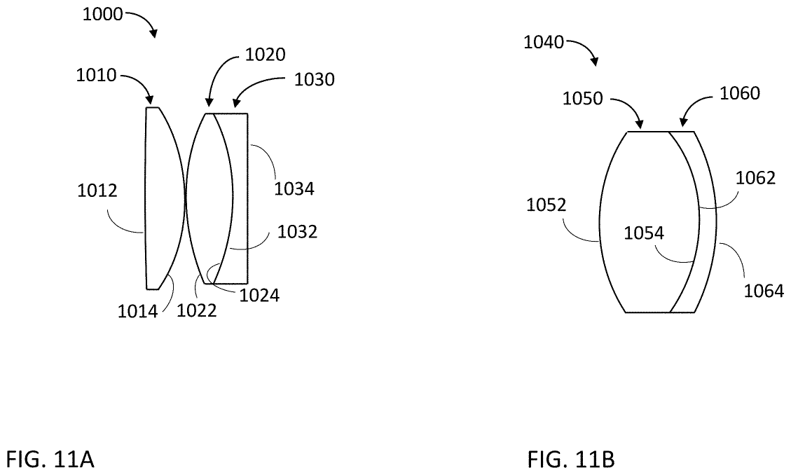

In some embodiments, objective lenses 328 may be configured to focus light from source components on the subject's eye and focus light from the subject's eye toward the appropriate detection components. In some embodiments, objective lenses 328 may include a plurality of plano-concave (PCV), plano-convex (PCX), and biconcave lenses. For example, objective lenses 328 may include two opposite-facing PCX lenses with a PCV lens and a biconcave between the PCX lenses. In some embodiments, objective lenses 328 may include an achromatic doublet. For example, the achromatic doublet can include a biconvex (BCX) lens and a meniscus negative lens. In some embodiments, one or more of the lenses of objective lenses 318 can include an aspheric surface, which provides improved image sharpness. For example, the aspheric surface can be a rear surface of the achromatic doublet. Objective lenses are further discussed in connection with FIGS. 11A and 11B below, in accordance with some embodiments.

In some embodiments, fixation components 330 may be configured to transmit fixation light toward the subject's eye to display a visible fixation object. In FIG. 5B, fixation components 330 include fixation display 332, fixation lenses 334, and pupil relay 336. For example, fixation display 332 may be configured to display a visible fixation object, and fixation lenses 334 may be configured to focus fixation light from fixation display 332 on the subject's eye, such as the subject's pupil via pupil relay 336. In some embodiments, fixation display 332 may be configured to display the fixation object in various positions to cause the subject's eye to move in particular directions when the subject is directed (e.g., by an audio queue from the imaging and/or measuring apparatus and/or a technician) to track the fixation object.

In some embodiments, fluorescence detection components 340 may be configured to receive fluorescent light from the subject's eye reflected via fluorescence dichroic 324. In FIG. 5B, fluorescence detection 340 components include machine vision (MV) lenses 342 and fluorescence sensor 344. In some embodiments, MV lenses 342 may be configured to provide diopter compensation for received light from the subject's eye. In some embodiments, MV lenses 342 may be adjustable to provide adjustable diopter compensation. For example, MV lenses 342 may be configured as part of a diopter flexure assembly described further herein. As shown in FIG. 5B, MV lenses 342 may be configured to be adjusted by diopter motor 360. For example, diopter motor 360 may be configured to adjust a positioning of MV lenses 342 to adjust the diopter compensation provided by MV lenses 342.

In some embodiments, fluorescence sensor 344 may be configured to capture fluorescent light to perform fluorescence imaging. For example, fluorescence sensor 344 may be an integrated device configured to perform fluorescence lifetime imaging, fluorescence spectral imaging (e.g., autofluorescence spectral imaging), and/or fluorescence intensity imaging. In the example of fluorescence lifetime imaging, fluorescence sensor 344 may be configured to receive incident fluorescent emissions and determine luminance lifetime information of the fluorescent emissions. In the example of fluorescence spectral imaging, fluorescence sensor 344 may be configured to determine luminance wavelength information of the fluorescent emissions. In the example of fluorescence intensity, fluorescence sensor 344 may be configured to determine luminance intensity information of the fluorescent emissions. In some embodiments, fluorescence sensor 344 may have one or more processors integrated thereon, and/or may be coupled to one or more processors onboard the imaging and/or measuring apparatus and configured to provide lifetime, wavelength, and/or intensity information to the processor(s) for image formation and/or measurement.

In some embodiments, white light detection components 350′ may be configured to capture white light received from the subject's eye to produce one or more images and/or measurements of the subject's eye. As shown in FIG. 5B, white light detection components 350′ may include MV lenses 352 and a white light camera 353. In some embodiments, MV lenses 352 may be configured in the manner described for MV lenses 342. For example, in FIG. 5B, MV lenses may be configured to provide adjustable diopter compensation, and diopter motor 360 may be configured to adjust the diopter compensation provided by MV lenses 352 by adjusting a positioning of MV lenses 352. In some embodiments, diopter motor 360 may be configured to adjust MV lenses 342 and 352 independently of one another. For example, diopter motor 360 may be configured to generate motion in two or more orthogonal directions, such as along an axial direction and rotationally about the axial direction, with motion along one direction configured to adjust MV lenses 342 and with motion along another direction configured to adjust MV lenses 352. In some embodiments, diopter motor 360 may be configured to automatically adjust MV lenses 342 and/or 352 based on signals received from one or more processors onboard the imaging and/or measuring apparatus. It should be appreciated that, in some embodiments, diopter motor 360 may be alternatively or additionally configured to adjust MV lenses of fixation components 330.

In some embodiments, white light camera 354 may be configured to produce one or more images and/or measurements of the subject's eye using light received via MV lenses 352. In some embodiments, white light camera 354 may include a color camera. In some embodiments, white light camera 354′ may include a monochrome camera. In some embodiments white light camera 354′ may be configured to output image and/or measurement information to one or more processors onboard the imaging and/or measuring apparatus.

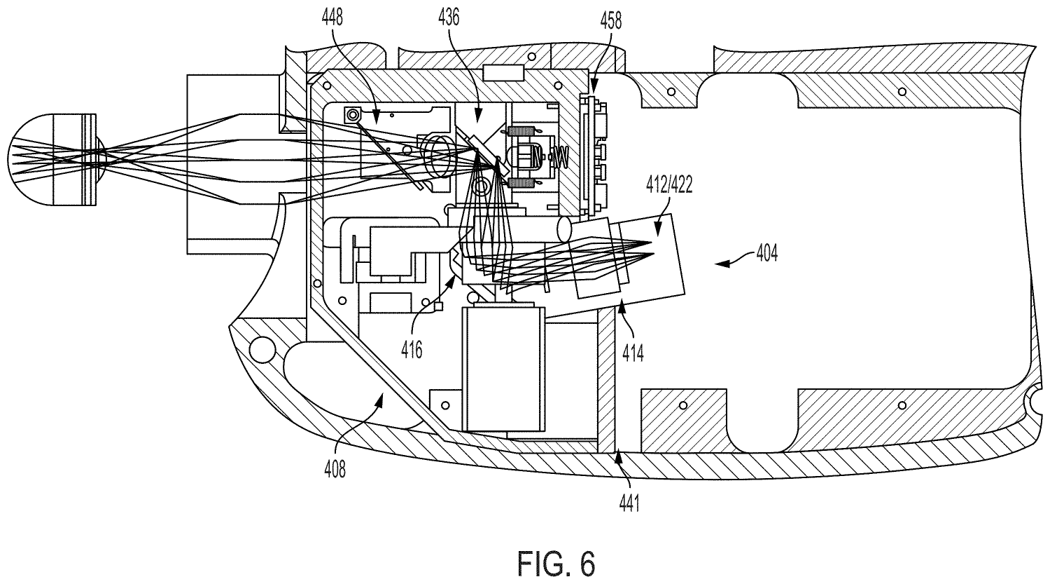

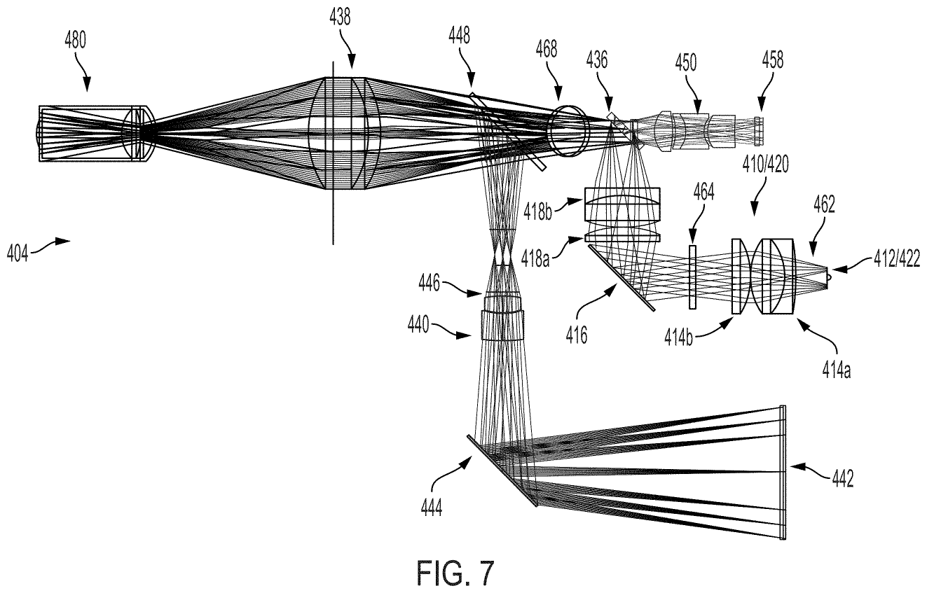

FIG. 6 is a top view of a portion of the imaging and/or measuring apparatus of FIG. 2 showing the white light and fluorescence detection components 404 of the imaging and/or measuring apparatus, according to some embodiments. In FIG. 6, white light and fluorescence imaging components 404 include light sources 412 and 422, collimating lens 414, mirror 416, illumination mirror 436, fixation beamsplitter 448, and camera 458. FIG. 6 also shows a fixation display mount 441, where a fixation display (e.g., 442 in FIG. 7) may be positioned, in some embodiments, and housing member 408 supporting white light and fluorescence imaging components 404.

In some embodiments, light sources 412 and 422 may be white light and/or fluorescence light sources, respectively. For example, light sources 412 and 422 may be configured to generate light for illuminating and/or exciting molecules in a subject's eye via collimating lens 414. In some embodiments, mirror 416 may be configured to reflect light form light sources 412 and 422 toward illumination mirror 436. In some embodiments, fixation beamsplitter 448 may be configured to reflect light form the fixation display toward the subject's eye. In some embodiments, illumination mirror 436 may be configured to transmit light received form the subject's eye to camera 458. For example, in some embodiments, illumination mirror 436 may be configured to transmit light received from the subject's eye to camera 458. For example, in some embodiments, illumination mirror 436 may have an aperture positions such that light received from light sources 412 and 422 reflects off portions of illumination mirror 436 and light received from the subject's eye passes through the aperture, as further described above in connection with FIG. 5B.

It should be appreciated that, in some embodiments, imaging apparatuses described herein may have fewer and/or different combinations of imaging components than shown in FIG. 6. For example, an imaging apparatus may have only white light and/or fluorescence imaging components. In another example, an imaging apparatus may have white light, fluorescence, OCT and/or IR imaging components position on a same side of the imaging apparatus, as aspects of the technology described herein are not limited in this respect.

The inventors have appreciated that providing a portable apparatus for imaging and/or measuring of a subject's fundus presents challenges. Optical systems may be highly sensitive to the alignment of their optical components, even misalignments smaller than a millimeter may render an imaging and/or measuring system unsuitable for diagnosing the condition of a subject. Therefore, optical systems are conventionally designed to be stationary and, in some cases, mounted to dampened optical breadboards to reduce the impact of vibrations. Apparatuses that include multiple and/or shared optical paths compound these challenges as the alignment of the optical components in each path may affect the functionality of the apparatus.

In some embodiments, an imaging and/or measuring apparatus can include multiple optical paths (e.g., detection, illumination, and fixation) that utilize sample components configured between an objective lens and a detector to combine and/or separate the different optical signals (e.g., illumination light and/or fixation light), such that the light shares a common optical path to and/or from the subject's eye. The sample components used to combine and/or separate the different optical paths may have a significant impact on the overall power efficiency, or imaging and/or measurement function of the device. For example, beamsplitters may determine what portion of light is transmitted versus reflected. Dichroics may determine what wavelengths are transmitted versus being blocked from a detector. Similarly, the specific mirrors and lenses may introduce distortions into the illumination, fixation, and detection optical paths. The inventors have developed illumination optical components to reduce the light being scattered to the detector while illuminating a subject's eye. One example of sample components configured between an objective lens and a detector to support multiple optical paths is shown in FIG. 7.

FIG. 7 is a schematic view of the source (410), fixation (440), and detection components (450) of FIG. 6, according to some embodiments. In FIG. 7, white light and fluorescence imaging components 404 include source components 410 and 420, sample components, fixation components 440, and detection components 450. In some embodiments, source components 410 and 420 may be configured to generate and transmit light to a subject's eye via the illumination and sample components, and detection components 450 may be configured to receive light from the subject's eye and capture an image using the received light. In some embodiments, fixation components 440 may be configured to display a fixation object to the subject before, during, and/or after imaging.

In FIG. 7, source components 410 and 420 include light sources 412 and 422, collimating lenses 414a and 414b, mirror 416, and focusing lenses 418a and 418b. In some embodiments, light sources 412 and 414 may be white light and fluorescence light sources, respectively. Collimating lenses 414a and 414b are shown in FIG. 7 as an achromatic lens and a plano-convex lens, respectively, which may be configured to collimate light from light sources 412 and 422. Mirror 416 is shown in FIG. 7 configured to reflect light from light sources 412 and 422 toward illumination mirror 436 of source components 430. Focusing lenses 418a and 418b are shown in FIG. 7 as a plano-convex lens and an achromatic lens, respectively, which may be configured to focus light from light sources 412 and 422 on at least a portion of illumination mirror 436. In FIG. 7, focusing lenses 418a and 418b are shown focusing light on at least two portions of illumination mirror 436 without transmitting light to a center of illumination mirror 436.

In FIG. 7, the sample components include illumination mirror 436 and objective lens 438. In some embodiments, illumination mirror 436 may be configured to reflect light from source components 410 and 420 toward the subject's eye. In some embodiments, objective lens 438 may be configured to focus light from illumination mirror 436 on the subject's eye and to focus light received from the subject's eye on illumination mirror 436. Illumination mirror 436 may be further configured to transmit light received from the subject's eye via objective lens 438 toward detection components 450.

In FIG. 7, fixation components 440 include fixation display 442, fixation mirror 444, fixation focusing lenses 446, and fixation dichroic 448. In some embodiments, fixation display 442 may be configured to display a fixation object to the subject's eye before, during, and/or after imaging. In some embodiments, fixation mirror 444 may be configured to reflect fixation light from fixation display toward fixation dichroic 448, which may be configured to provide the fixation light to the subject's eye along an illumination path along which the subject's eye is illuminated with light from source components 410 and 420. In some embodiments, fixation focusing lenses 446 may be configured to focus the fixation light on the subject's eye. In FIG. 7, fixation components 440 share at least part of an optical path with source components 410 and 420, as sample components 430 convey fixation light and illumination light via objective lens 438. In FIG. 7, detection components 450 include camera 458 and lenses configured to focus light received from the subject's eye on camera 458.

IV. Spatial Filtering Components

As discussed above, the inventors recognized that certain portions of a subject's eye produce undesired reflections when illuminated during imaging, and the reflections can degrade the quality of images captured when the eye is illuminated. For example, the cornea and/or iris of the subject's eye may produce very bright reflections that can be transmitted to the imaging sensor along with desired reflections, from portions of interest of the subject's eye. The desired reflections may be less bright than the reflections from cornea and/or iris reflections, thereby degrading image quality. The inventors have further recognized that the surfaces of the optical components of the apparatus may additionally produce reflections that scatter light to the detector decreasing the contrast and/or resolution of images and measurements. the inventors have appreciated that, for some applications, illumination components (e.g., source components) for illuminating a subject's eye without the use of polarizing optical components may increase the contrast and decrease the cost of an imaging and/or measuring apparatus. As also discussed above, the inventors have further appreciated that, for some applications, illumination components (e.g., source components) for illuminating a subject's eye without the use of polarizing optical components may increase the energy efficiency and decrease the cost of an imaging and/or measuring apparatus.

To address these problems, the inventors developed techniques for modifying the illumination profile to suppress reflections, that would otherwise decrease image quality when transmitted to the detector, by using spatial filters and appropriate lenses. For some applications, the techniques for modifying the illumination profile to suppress reflections, that would otherwise decrease image quality when transmitted to the detector, may be used without polarizing components. In some embodiments, light source 412 and/or 422 may be configured to provide the illumination light and lenses 414a and 414b and 418a and 418b and objective lens 438 may be configured to transmit the illumination light to a subject's eye 480. In some embodiments, light source 412 and/or 422 may be configured to transmit the illumination light through plates 462 and 464 (FIGS. 8C and 8D, respectively) as described further herein. In some embodiments, illumination mirror 546 (FIG. 8E) may be configured to block at least some light reflected from the subject's eye 480 from reaching detection components 450.

In some embodiments, using an annular light source increases the energy efficiency of the apparatus. When using spatial filters, such as plates 462 and 464, light that does not match the desired illumination profile is blocked or attenuated and optical power is lost. By generating light using an annular light source, such as those illustrated in FIGS. 8A and 8B, a larger portion of the generated light is transmitted through the spatial filter unattenuated, conserving a greater portion of the optical power and increasing power efficiency.

FIG. 8A is a front view of light source components, including light sources 412a and 412b, that may be included in white light and fluorescence imaging components 404, according to some embodiments. In FIG. 8A, light sources 412a and 412b are among a group of light sources arranged in a ring. In some embodiments, the light sources may be white and/or IR LEDs. In some embodiments, the light sources may be independently controllable and configured to illuminate portions of the subject's eye. For example, each light source or each of multiple groups of light sources may be configured to illuminate a respective portion of the subject's eye, or ones of the light sources may be configured to overlap in illumination over various portions of the subject's eye. In some embodiments, white light and fluorescence imaging components 404 may be configured to selectively illuminate one or more of the light sources to selectively illuminate one or more portions of the subject's eye.

FIG. 8B is a front view of alternative light source components, including light sources 412a, 412b, 422a, and 422b, that may be included in white light and fluorescence imaging components 404, according to some embodiments. The light sources of FIG. 8B may be configured in the manner described for the light sources of FIG. 8A. In some embodiments, light sources 412a, and 412b may be white light sources and light sources 422a and 422b may be IR light sources.

It should be appreciated that, in some embodiments, the light sources may alternatively or additionally include light sources positioned at the center of the rings illustrated in FIGS. 8A and 8B.

FIG. 8C is a front view of a plate 462 of white light and fluorescence imaging components 404, according to some embodiments. In FIG. 8C, plate 462 includes outer portion 462a and inner portion 462c with annular window 446b between outer portion 462a and obscuration 462c. In FIG. 7, plate 462 is positioned between light sources 412/422 and collimating lenses 414a/414b. In some embodiments, light sources 412 and 422 may be configured to transmit light through annular window 462b to collimating lenses 414a and 414b. In some embodiments, collimating lenses 414a and 414b, focusing lenses 418a and 418b, and/or objective lens 438 may be configured to light transmitted through annular window 462b to one or more portions of the subject's eye.

In some embodiments, as an alternative or in addition to the light sources of FIGS. 8A and 8B and/or the plate 462 of FIG. 8C, white light and fluorescence imaging components 404 may include an illumination control device, such as a digital micromirror device and/or a digital light projector configured to selectively illuminate one or more portions of the subject's eye as described herein for the light sources of FIGS. 8A and 8B and the LCD screen. For example, the illumination control device may be configured to receive illumination light from one or more light sources 412 and/or 422 and selectively direct the illumination light to one or more portions of the subject's eye.

FIG. 8D is a front view of a plate 464 with an obscuration of white light and fluorescence imaging components 404, according to some embodiments. As shown in FIG. 8D, plate 464 includes obscuration 464a. In FIG. 7, plate 464 is positioned between collimating lenses 414a and 414b and mirror 416. In some embodiments, light from at least some of the light sources may be blocked from reaching the subject's eye by obscuration 464a. The inventors have recognized that the objective lens that focuses the transmitted light on the subject's eye may cause undesired reflections to reach the imaging sensor when certain portions of the objective lens are illuminated. To address this problem, a spatial filter, such as obscuration 464a may be positioned at a conjugate focal plane to one of the surfaces of the objective lens, thereby blocking the illumination light from reaching at least some portions of the objective lens, thereby reducing undesired reflections reaching the imaging sensor.

In some embodiments, the positioning of the spatial filter may modify the illumination profile on a surface of the objective, such that a portion of the surface is not illuminated or is illuminated with a diminished intensity when compared with the illumination profile in absence of the spatial filter.

In some embodiments, the positioning of the spatial filter may be configured to block and/or attenuate light transmitted at specific angles from being transmitted to the objective lens. The spatial filter may be configured to obscure rays propagating at specific angles that could reflect off a surface of the objective lens into the detection components. For example, the spatial filter may be configured to block or obscure rays traveling parallel to the axis at a conjugate focal plane of a surface of the objective lens.

In some embodiments, multiple spatial filters may be included and configured at conjugate focal planes for different surfaces of the objective lens. The spatial filters may be sized to account for a magnification of the spatial filter by other lenses in the optical path. Additionally, the spatial filters may have any suitable shape. In some embodiments, the spatial filter may be circular, rectangular, or may include a periodic pattern. In some embodiments, the spatial filter may be distorted to compensate for the distortions of the optical components.

FIG. 8E is a front view of illumination mirror 463 of white light and fluorescence imaging components 404, according to some embodiments. As shown in FIG. 8E, illumination mirror 436 includes aperture 436a. In some embodiments, focusing lenses 418a and 418b may be configured to focus light received from light sources 412 and 422 on portions of illumination mirror 436 other than aperture 436a such that the light is reflected toward the subject's eye. In some embodiments, objective lens 438 may be configured to focus light received from the subject's eye on aperture 436a such that the light is reflected toward the subject's eye. In some embodiments, objective lens 438 may be configured to focus light received from the subject's eye on aperture 436a such that the received light is transmitted to detection components 450 (e.g., to camera 458) through aperture 436a. In some embodiments, objective lens 438 may be configured to focus light received from the subject's eye on aperture 436a such that the received light is transmitted to detection components 450 (e.g., to camera 458) through aperture 436a. In some embodiments, mirror 436 may block reflections from undesired portions of the eye from reaching detection components 450. For example, substantially all of the undesired reflections may reflect off of parts of mirror 436 other than aperture 436a.

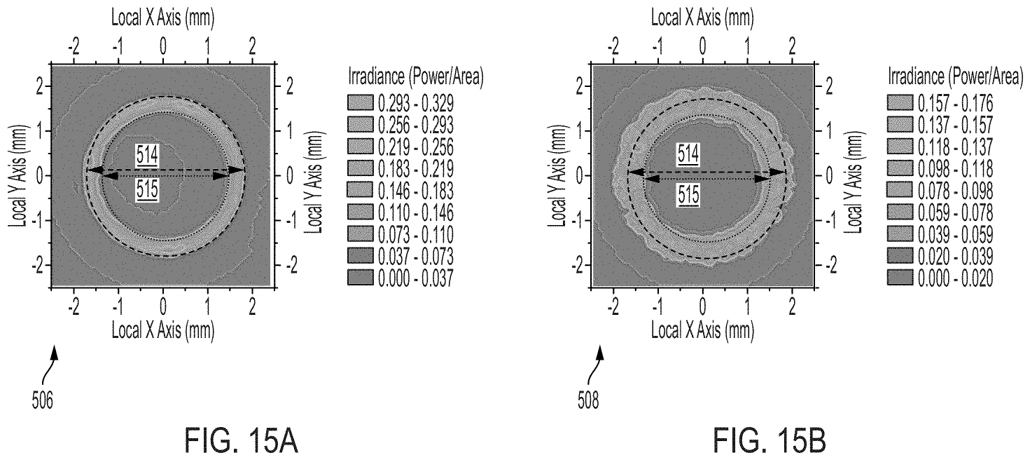

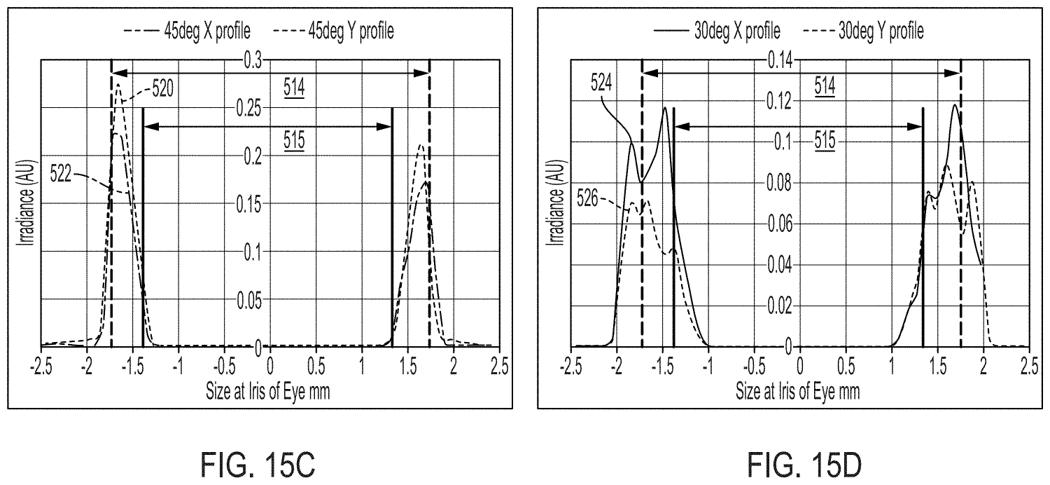

Exemplary illumination profiles that may be generated by the components described herein are illustrated in FIGS. 15A-D, 16A, and 16B, described further below.

V. Wide Angle Field of View and Interchangeable Optical Components

As described above, the inventors have appreciated that an apparatus with a wide-angle field of view may be advantageous in some embodiments for providing a wider field of view of the tissue for analysis and/or making health determinations. The inventors have developed objective lens and corresponding sample, detect, illumination, and fixation optical components to overcome the challenges discussed above regarding providing a portable, cost effective, and energy efficient apparatus for wide angle imaging and/or measuring a subject's retina fundus. In some embodiments, the imaging and/or measuring apparatus includes a lens capable of providing a 30 degree field of view of a subject's eye, a lens capable of providing a 45 degree field of view of a subject's eye, or a lens capable of providing a 60 degree field of view of a subjects eye. Exemplary objective lenses that may be configured with the imaging and/or measuring apparatus are further described below in connection with FIGS. 11A and 11B.

In some applications, the apparatus may be used to collect wide-angle field of view images and/or measurements without the use of an optical scanner. Thereby reducing the number of moving parts, energy consumption, and cost of production.

As described above, the inventors have appreciated that an apparatus that can provide different fields of view may provide advantages for detecting and/or diagnosing a subject based on images and/or measurements of the subject's eye by providing different magnifications and/or resolutions. The imaging and/or measuring apparatus can be configured to transmit and/or receive light having different fields of view by using different objectives. While the field of view may be changed by adjusting the active area of the detector or field stops (i.e., apertures that change the field of view), changes to the field of view that do not also result in a change of magnification will not provide the same advantages as adjusting the field of view by changing the objective lens. Increasing, or decreasing, the field of view by changing the objective also changes the magnification of the apparatus which may be advantageous for detecting and/or diagnosing a subject's eye. However, as described above, lenses are not interchangeable. Swapping one lens for another will change the convergence and divergence of light transmitted through the optics. Accordingly, changing one optical component, such as an objective lens, may require that other optical components swapped or reconfigured to produce suitable images and/or measurements. Due to the sensitivity of alignment, the repositioning of optical components may a time-consuming task, that may require specialized training as misalignment of components may render an apparatus unsuitable for imaging and/or measuring.

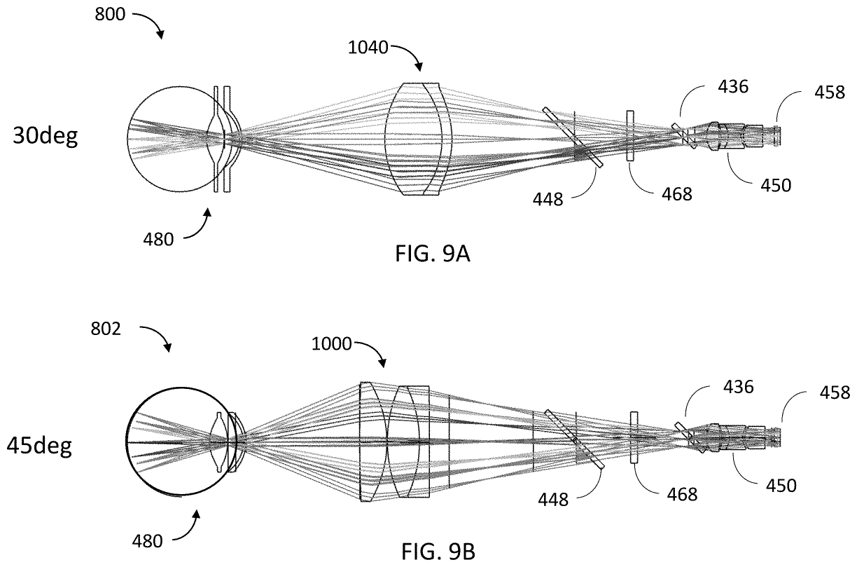

The inventors having recognized the challenges above, have developed a portable apparatus that supports different objectives to provide different fields of view that may be readily changed, without the requiring time-consuming adjustments or specialized training, where each of the optical components is designed and configured to support the different objectives with minimal or no adjustment. FIGS. 9A and 9B illustrate optical components designed to support different objectives, in accordance with some embodiments.

FIG. 9A illustrates sample and detect components 800 of an imaging and/or measuring apparatus, in accordance with some embodiments. FIG. 9 illustrates subject's eye 480, and sample and detect components 800 including objective lens 1040, fixation beamsplitter 448, dichroic 468, holed mirror 436, MV lens 450 and detector 458. Light transmitted from the eye is captured by objective lens 1040 and transmitted by objective lens 1040 through sample components 448, 468, and 436 to detect components 450 and 458.

Sample components may modify the intensity of the transmitted light, in accordance with some embodiments. For example, a beamsplitter, such as fixation beamsplitter 448, may reflect a portion of the transmitted light reducing the intensity of the transmitted light. In some embodiments, the beamsplitter may reflect less than 10% of the light received by the beamsplitter. In other embodiments, the beamsplitter may be a 10:90 beamsplitter (i.e., 10% reflective and 90% transmissive), a 30:70 beamsplitter, a 50:50 beamsplitter, a 70:30 beamsplitter, or a 90:10 beamsplitter. In some embodiments, fixation beamsplitter 448 may be configured as fixation beamsplitter 326, as described above in connection with FIG. 5B. Other beamsplitters may be used, as aspects of the technology described herein are not limited in this respect.

Sample components may modify the spectral bandwidth of the transmitted light, in accordance with some embodiments. For example, a dichroic may be configured to reflect some wavelengths of light and transmit other wavelengths of light. Dichroic 468 may be configured as a long pass filter, a short pass filter, a bandpass filter, or a notch filter. Dichroic 468 may be implemented in the same way as fluorescence dichroic described above in connection with FIG. 5B. Other dichroics may also be used as aspects of the technology described herein are not limited in this respect.

As referenced above in the description of FIG. 8E, holed mirror 436 may be configured to obscure a portion of the light received from the subject's eye. For example, the aperture of holed mirror 436 may have a diameter smaller than the beam width of the light transmitted through the aperture of the holed mirror. Portions of the light received from the subject's eye that are not transmitted through the aperture of the holed mirror will be obscured from being transmitted to the detection components. In some embodiments, holed mirror 436 is configured near a conjugate plane of the optical components to affect the numeric aperture of the imaging and/or measuring apparatus. In other embodiments, the holed mirror may have a diameter larger than the beam width of the light transmitted through the aperture of the holed mirror.

The apparatus may not include each of the sample components, in accordance with some embodiments. For example, dichroic 468 may be excluded in embodiments without a separate fluorescence detector. As such, in some embodiments, the apparatus may be configured to use the same detector for capturing white light images as performing fluorescence detection. In other embodiments, the apparatus may not be configured to perform fluorescence imaging and/or measurements. In yet other embodiments, the fluorescence imaging and/or measuring components may be configured as part of a separate optical path for performing imaging and/or measurements on the subject's second eye (not pictured).

Detect components are configured to image and/or measure at least a portion of the light received from subject's eye 480. MV lens 450 may be configured for diopter compensation as described above with reference to MV lens 352 of FIG. 5A. In some embodiments, MV lens 450 transmits light received through the aperture of the holed mirror to detector 458. For example, MV lens 450 may transmit light to form an in-focus image of a subject's retina fundus on detector 458.

Detector 458 is configured to detect a field of view of the subject's retina fundus. As described above, the detector may have a square detection region or a rectangular detection region. The detection region may be an array of detectors disposed in the xy-plane (perpendicular to the propagation of light impinging on the detector). In some embodiments where the detection region is square, the array of detectors may for a detection region having the same length in the x-direction as in the y-direction. In other embodiments, the array of detectors have form a detection region having a different length in the x-direction as in the y-direction.

The field of view detected by detector 458 may be the same along a first dimension of the detector as along a second dimension of the detector. In other embodiments, the field of view detected by detector 458 may be wider in a first dimension than in a second dimension. In some embodiments, the detector is a square detector 3.76 mm long in an x-direction and 3.74 mm long in a y-direction.

As described above, the effective focal length and the detector size are related to the field of view and the effective focal length will depend on each of the optical components that affect the convergence and divergence of the transmitted light. When the apparatus is configured with objective lens 1040, as illustrated in FIG. 9A, the apparatus may produce images and/or measurements with a field of view between 25 and 35 degrees on the detector. The objective lens 1040 is further described below in connection with FIG. 11B.

The same components may be configured with a different objective lens to produce a different field of view. FIG. 9B is a diagram of another configuration of detection components 802, according to some embodiments. FIG. 9B illustrates the same components as FIG. 9A with the exception of the objective lens. In FIG. 9B, objective lens 1000 is used instead of objective lens 1040. Sample and detect components 802 may be the same as sample and detect components 800. For example, the same fixation beamsplitter 448, dichroic 468, and holed mirror 436 and the same detection components 450 and 458 may be used with objective 1000 to perform imaging or measuring of subject's eye 480. The apparatus may produce images and/or measurement switch a field of view between 40 and 50 degrees on the detector. The objective lens 1000 is further described below in connection with FIG. 11A.

As described above, optical apparatuses with multiple optical paths present further challenges for alignment. For example, the objective lens transmits light from the illumination and fixation optical components to the subject's eye, thus the transmission of light to the subject's eye will depend on the configuration of the illumination components, fixation components, and the objective lens.

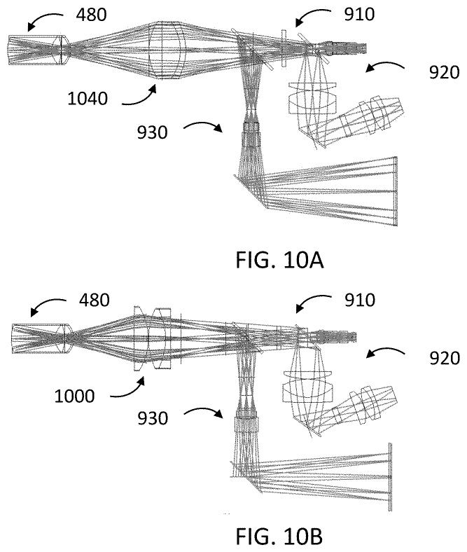

FIG. 10A is a schematic view of a configuration of the illumination 920, fixation 930, and detection components 910, according to some embodiments. The illumination, fixation, and detection components illustrated in FIG. 10A are configured to provide images and/or measurements with approximately a 30 degree field of view of a subject's eye 480. Objective lens 1040 transmits light received from both illumination optical components 920 and fixation optical components 930 to the subject's eye 480. Further, objective lens 1040 receives light from an illumination portion of the subject's eye 480 and transmits the received light to detection optical components 910.

Illumination optical components are configured to transmit illumination light to objective lens 1040. Illumination light is further transmitted to the subject's eye by objective lens 1040. Objective lens 1040 is configured such that when the illumination light is transmitted through the pupil of a subject's eye the light illuminates a portion of the subject's retina fundus that includes the field of view to be imaged and/or measured by the apparatus.

In some embodiments, fixation optical components are configured to transmit fixation light to objective lens 1040 such that when fixation light is transmitted to the subject's eye, the fixation light is transmitted to the subject's retina fundus to indicate an alignment of the subject's eye relative to the field of view produced by the objective lens 1040 and the detection optical components 910.

FIG. 10B is a schematic view of another configuration of the illumination 920, fixation 930, and detection components 910, according to some embodiments. The illumination, fixation, and detection components illustrated in FIG. 10B are configured to provide images and/or measurements with approximately a 45 degree field of view of a subject's eye 480. Objective lens 1000 transmits light received from both illumination optical components 920 and fixation optical components 930 to the subject's eye 480. Further, objective lens 1000 receives light from an illumination portion of the subject's eye 480 and transmits the received light to detection optical components 910. Objective lens 1000 is configured such that when the illumination light is transmitted through the pupil of a subject's eye the light illuminates a portion of the subject's retina fundus that includes the field of view to be imaged and/or measured by the apparatus.

In some embodiments, the illumination optical components, fixation optical components, and detection optical components used with objective lens 1040, as illustrated in FIG. 10A, are also used with objective lens 1000, as illustrated in FIG. 10B. The detection optical components 910 may be configured to produce a first field of view when used with objective lens 1040 and a second field of view when used with objective lens 1000. The detection optical components may be any of the detection optical components as described herein. In some embodiments, the apparatus will produce a magnification of 0.27 when configured with objective 1000 and a magnification of 0.41 when configured with objective 1040.

The illumination optical components may be configured to illuminate a portion of the eye including the first field of view when used with objective lens 1040 and may be configured to illuminate a portion of the eye including the second field of view when used with objective lens 1000. Additionally, the fixation optical components may be configured to transmit light to the subject's retina fundus to indicate an alignment of the subject's eye relative to the first field of view when used with objective lens 1040 and may be configured to transmit light to the subject's retina fundus to indicate an alignment of the subject's eye relative to the second field of view when used with objective lens 1000.

The inventors have appreciated that the number of lens elements included with a lens may improve the optical quality of the image and/or measurement but will also increase the cost and complexity of manufacturing. The inventors have designed cost effective objective lenses that include multiple lens elements (e.g., doublet, triplet, etc.) to provide sufficient optical quality for imaging and/or measuring. The inventors have further appreciated that the eye itself includes a flexible lens tissue that affects the convergence and divergence of light transmitted to or reflected from the retina fundus. Therefore, for accurate imaging and/or measuring of the eye, optical components configured to transmit and/or receive light from the eye should account for the curvature of the tissue itself. Exemplary objective lenses are described below in connection with FIGS. 11A and 11B, in accordance with some embodiments.

FIG. 11A is a diagram of an objective lens 1000, according to some embodiments. The objective lens 1000 may be a triplet lens capable of producing a field of view between 40 and 50 degrees when configured with detection components, as described herein. In some embodiments, the triplet lens includes lens elements 1010, 1020, and 1030. Lens element 1010 includes surfaces 1012 and 1014. Lens element 1020 includes surfaces 1022 and 1024. Lens element 1030 includes surfaces 1032 and 1034. In some embodiments, lens element 1010 is formed from glass having a refractive index of approximately 1.88 and an Abbe number of approximately 40 (e.g., S-LAH58 glass) and the center of surface 1012 and the center of surface 1014 are separated by a thickness of approximately 5.5 mm. Lens element 1020 is formed from glass having a refractive index of approximately 1.57 and an Abbe number of approximately 56 (e.g., S-BAL14 glass). The center of surface 1022 and the center of surface 1024 are separated by a thickness of approximately 6.5 mm. Lens element 1030 is formed from glass having a refractive index of approximately 1.9 and an Abbe number of approximately 19. The center of surface 1032 and the center of surface 1034 are separated by a thickness of approximately 2 mm. The radius of curvature of each lens element are described below in Table 1, in accordance with some embodiments.

TABLE 1
Objective lens 1000 lens surfaces and their radius of curvature.

| Lens Surface | Radius (mm) |
|---|---|
| 1012 | 306.337 |
| 1014 | −22.439 |
| 1022 | 26.736 |
| 1024 | −25.724 |
| 1032 | −25.724 |
| 1034 | −1022.307 |

In some embodiments, at least one of the lens elements includes an aspherical curvature. The curvature of an aspheric lens is given by:

$$z ( r ) = \frac{{r}^{2}}{R ( 1 + \sqrt{1 - ( 1 + κ ) \frac{{r}^{2}}{{R}^{2}}} )} + {α}_{4} {r}^{4} + {α}_{6} {r}^{6} + {α}_{8} {r}^{8} + {α}_{10} {r}^{10} + …$$

where r is the distance from the optical axis, R is the radius of curvature K is the conic constant αₙ is the n^{th} order term. In some embodiments, objective lens 1000 includes at least one aspherical surface. For example, the curvature of lens element 1010 and the lens element 1030 may include higher order terms that described the curvature of the aspherical surfaces. Table 2 illustrates higher order terms that describe the curvature of lens surfaces 1014 and 1034, in accordance with some embodiments.

TABLE 2
Approximate aspherical surface lens terms.

| | 4^{th} Order | 6^{th} Order | 8^{th} Order | 10^{th} Order |
|---|---|---|---|---|
| Lens Surface | Term | Term | Term | Term |
| 1014 | 4.3E−06 | −1.12E−8 | 1.9E−10 | 1.16E−13 |
| 1034 | 7.34E−6 | −3.67E−8 | 2.31E−12 | −2.25E−12 |

FIG. 11B is a diagram of another objective lens 1040, according to some embodiments. The Objective lens may be a double lens capable of producing a field of view between 25 and 35 degrees when configured with detection components, as described herein. In some embodiments, the double lens includes lens elements 1050 and 1060. Lens element 1050 includes surfaces 1052 and 1054. Lens element 1060 includes surfaces 1062 and 1064. In some embodiments, lens element 1050 is formed from glass having a refractive index of approximately 1.50 and an Abbe number of approximately 82. The center of surface 1052 and the center of 1054 are separated by a thickness of approximately 12.0 mm. Lens element 1060 is formed from glass having a refractive index of approximately 1.85 and an Abbe number of approximately 24. The center of surface 1052 and the center of surface 1054 are separated by a thickness of approximately 2.1 mm. The radius of curvature of each lens element are described below in Table 3, in accordance with some embodiments.

TABLE 3
Objective lens 1040 lens surfaces and their radius of curvature.

| Lens Surface | Radius (mm) |
|---|---|
| 1052 | 19.327 |
| 1054 | −18.157 |
| 1062 | −18.157 |
| 1064 | −18.750 |

In some embodiments, objective lens 1040 includes at least one aspherical surface. For example, the lens element 1060 may include higher order terms that describe the curvature of the aspherical surfaces. Table 4 illustrates higher order terms that describe the curvature of lens surface 1064, in accordance with some embodiments.

TABLE 4
Approximate aspherical surface lens terms.

| | 4^{th} Order | 6^{th} Order | 8^{th} Order | 10^{th} Order |
|---|---|---|---|---|
| Len Surface | Term | Term | Term | Term |
| 1064 | 6.95E−5 | −4.42E−7 | 4.57E−9 | −2.27E−11 |

In some embodiments, switching between a first objective lens to provide a first field of view and a second objective lens to provide a second field of view may include adjusting the position of detection optical components to compensate for differences between the first and second objective lens.

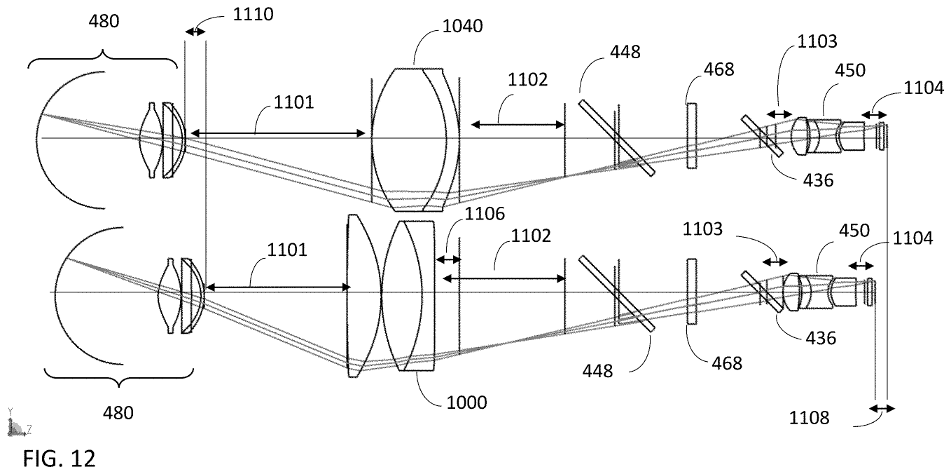

FIG. 12 is a diagram illustrating differences between a first configuration of detection components, which includes objective 1040, and a second configuration of detection components, which includes objective 1000, according to some embodiments. FIG. 12 illustrates sample and detection optical components configured for measuring and/or imaging a portion of a subject's eye. For example, when imaging and/or measuring, the subject's eye 480 and the objective are separated by a working distance 1101. When the apparatus is configured with objective lens 1000, the working distance 1101 is shorter by a distance 1110 relative to when the apparatus is configured with objective lens 1040. Accordingly, the distance 1102 between the objective lens and fixation beamsplitter 448 is longer by distance 1106 when used with objective lens 1000 relative to the configuration when used with objective lens 1040.

Sample optical components fixation beamsplitter, dichroic 468, and holed mirror 436 may be remain in the same position when switching between a first objective lens and a second objective lens. As described above, the sample optical components may be highly sensitive to changes in alignment, providing challenges to switching between objectives. The inventors have appreciated this challenge and designed sample optical components that may be configured for use with multiple objectives. In some embodiments, the placement of sample optical components are not modified when switching between objectives, providing straightforward switching between objectives.

In some embodiments, the detection components are shifted closer to the sample components when configured with objective lens 1000 relative to a configuration using objective 1040. For example, MV lens 450 is separated from holed mirror 436 by distance 1103 and the detector is separated from the MV lens 450 by distance 1104. The position of the detector when configured to be used with the first objective lens relative to the second objective lens may be shifted by distance 1108. Table 5 illustrates the approximate spacing between optical components illustrated in FIG. 12, in accordance with some embodiments.

TABLE 5
Changes in working distances and spacings
between sample and detect components.

| | Configured with | Configured with |
|---|---|---|
| Distance | Objective 1040 | Objective 1000 |
| 1101 | 30 mm | 23 mm |
| 1102 | 25 mm | 29 mm |
| 1103 | 3.9 mm | 2.5 mm |
| 1104 | 2.5 mm | 2.0 mm |

According to the embodiments illustrated in FIG. 12 and described in Table 5, the working distance 1101 is 7 mm shorter when the apparatus is configured with objective lens 1000 relative to objective lens 1040. Additionally, the spacing between the objective and the fixation beam splitter is longer by 4 mm, the spacing between the holed mirror 436 and the MV lens 450 is shorter by 1.4 mm, and the sensor is moved 1.8 mm closer to the holed mirror when the apparatus is configured with objective lens 1000 relative to objective lens 1040.

In some embodiment, a diopter motor, such as motor 360 (described above in connection with FIGS. 5A and 5B) may be configured to adjust MV lens 450 with a first resolution when configured with a first objective and a second resolution when configured with a second objective. For example, when configured with a wider field of view, diopter motor 360 may operate with a higher resolution and over a shorter travel range relative to operation of the diopter motor when configured with a narrower field of view. The inventors have recognized and appreciated that in some embodiments increasing the field of view may enable the diopter motor 360 to adjust the MV lens more quickly because the travel distance used for diopter compensation is shorter. Therefore, a wider field of view may provide faster image and/or measurement acquisition than a narrower field of view.

In some embodiments, when the apparatus is configured to produce a field of view between 25 and 35 degrees, the MV lens may move less than or equal to 2 mm, less than or equal to 1.5 mm, or less than or equal to 1 mm to provide ±8 diopter compensation. In other embodiments, when the apparatus is configured to produce a view of view between 40 and 50 degrees, the MV lens may move less than or equal to 0.8 mm, less than or equal to 0.6 mm, less than or equal to 0.5 mm, or less than or equal to 0.3 mm to provide ±8 diopter compensation.

In some embodiments, a motor may be configured to shift the position of the detect components when switching between objectives. For example, the diopter motor used to adjust MV lens 450 may be further configured to shift the position of detect components. In other embodiments, detect components may include a physical mechanism for manually shifting the position of the detect components when switching between objectives.

Additionally, switching between a first objective lens to provide a first field of view and a second objective lens to provide a second field of view may include adjusting the position of illumination optical components to compensate for differences between the first and second objective lens.

FIG. 13 is a diagram illustrating differences between a first configuration of illumination components 1200a and a second configuration of detections components 1200b, according to some embodiments. In some embodiments, the optical components between fold mirror 416 and holed mirror 436 may not be adjusted when switching between objective lenses. Spatial filter 464, collimating lens 414 and light source 412 may be shifted closer to the fold mirror 416. As shown in FIG. 13, spatial filter 464 is shifted a distance 1202, collimating lens 414 is shifted a distance 1204, and light source 412 is shifted a distance 1206 closer to fold mirror 416. In some embodiments, distance 1202 is 1.4 mm, distance 1204 is 1.7 mm, and distance 1206 is 0.4 mm. Table 6 illustrates lengths for distances 1201, 1203, and 1205, in accordance with some embodiments.

TABLE 6
Changes in spacings between illumination components.

| | Configured with | Configured with |
|---|---|---|
| Distance | Objective 1040 | Objective 1000 |
| 1201 | 14.45 | 13.02 |
| 1203 | 3.71 | 6.33 |
| 1205 | 6.21 | 7.41 |

In some embodiments, a motor or a physical mechanism may be included for shifting the position of the illumination components when switching between objective lenses, as described herein.

Switching between a first objective lens to provide a first field of view and a second objective lens to provide a second field of view may include adjusting the position of fixation optical components to compensate for differences between the first and second objective lens.

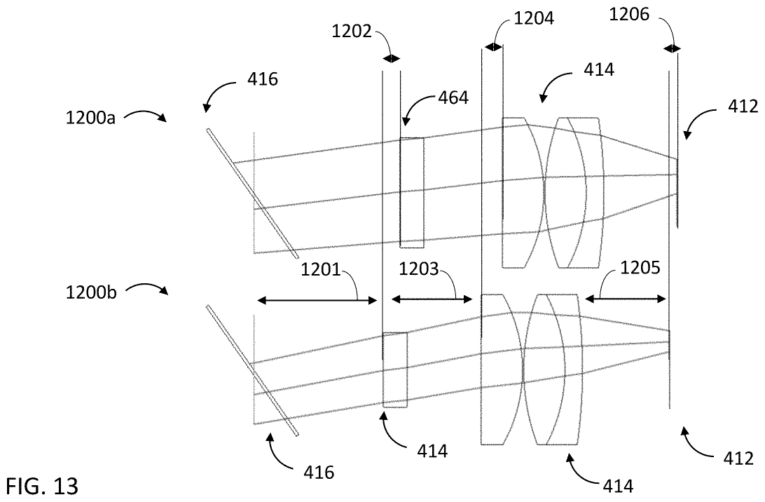

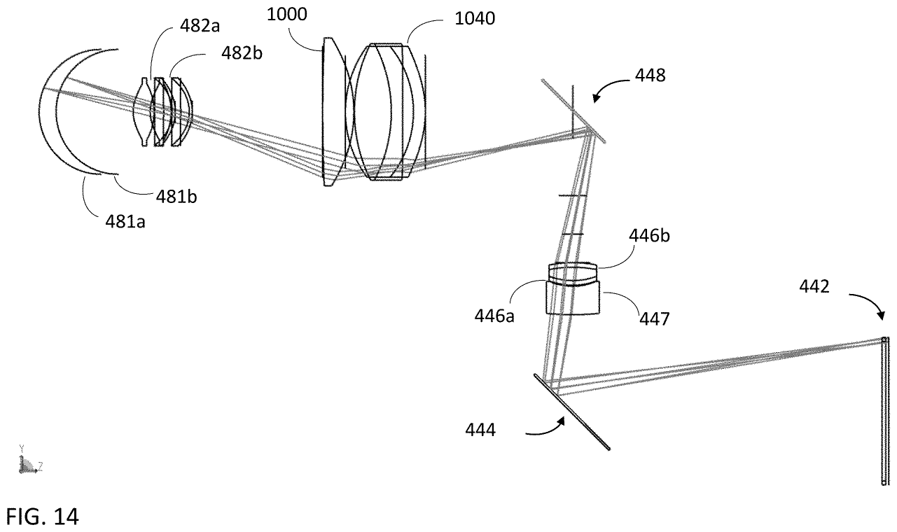

FIG. 14 is a diagram illustrating differences between a first configuration of fixation components, which are configured with objective 1040, and a second configuration of fixation components, which are configured with objective 1000, according to some embodiments. FIG. 14 illustrates fixation optical components, objective lenses, and a subject's eye in two different configurations overlaid with each other to illustrate the differences between the first configuration of the apparatus configured with objective lens 1040 to image and/or measure a subject's eye with a 25-35 degree of view, and a second configuration of the apparatus configured with objective lens 1000 to image and/or measure a subject's eye with a 40-50 degree field of view.

In some embodiments, fixation components include fixation display 442, fixation mirror 444, first fixation lens 446 and second fixation lens 447, fixation beamsplitter 448 configured to transmit light to an objective lens that transmits light to the subject's eye. In some embodiments, objective 1040 is used to transmit the fixation light through a subject's pupil 482a to the subject's retina fundus 481a. In other embodiments, objective lens 1000 is used to transmit the fixation light through a subject's pupil 482b to the subject's retina fundus 481b.

As illustrated in FIG. 14, the differences in working distance between objective lens 1000 and objective lens 1040 are reflected in the positions of the objectives and the positions of subject's eye 481a and 482a relative to subject's eye 481b and 482b.

In some embodiments, first fixation lens 446 may be located either at a first position as indicated by 446a or at a second position as indicated by 446b. For example, when the apparatus is configured with the fixation optical components configured to be used with objective lens 1000, the first fixation lens is disposed at position 446a. As another example, when the apparatus is configured with the fixation optical components configured to be used with objective lens 1040, the first fixation lens is disposed at position 446b. In some embodiments, position 446a is 0.8 mm closer to the second fixation lens 447 relative to position 446b.

The inventors have appreciated that due to the varying magnification that may be provided when switching between objective lenses, a fixation system that provides feedback to a subject by transmitting light to the subject's eye may be subjected to vignetting and/or field clipping. To provide fixation optical components that are configured to provide feedback to the subject by transmitting light to the subject's eye without vignetting or field clipping the inventors have developed fixation optical components that can be adjusted for use with different objective lenses to provide a field of view of a fixation display to a subject. In some embodiments, the region of the fixation display used to display the field of view may depend on the objective lens configured for imaging and/or measuring. For example, to provide feedback to the subject's eye, a field of view of the fixation display is used to display feedback to the subject that is transmitted using the fixation optical components and the objective lens to the subject's eye. When configured to provide feedback to a subject with an apparatus configured to provide an imaging and/or measuring field of view between 25 and 35 degrees, the field of view of the fixation display has a maximum diameter of 13 mm. When configured to provide feedback to a subject with an apparatus configured to provide an imaging and/or measuring field of view between 40-50 degrees, the field of view of the fixation display has a maximum diameter of 12.3 mm.

VI. Exemplary Illumination Profiles

In some embodiments, to image or measure a portion within the eye (e.g., the retina fundus) illumination from the imaging and/or measuring apparatus is needed. As described above, the inventors have recognized that illuminating a subject's retina fundus provides challenges. To transmit light into the eye, the light must pass through the pupil. Portions of the illumination light that do not pass through the pupil will scatter off other portions of the eye, such as the iris. Given the high reflectivity of the iris, light scattered by the iris can obscure light collected from within the eye due to the differential between the brightness of the light scattered by the iris relative to light scattered by the retina fundus. Furthermore, light transmitted to the eye that is not transmitted through the pupil will not contribute to the image or measurement of the retina fundus and accordingly may result in lost power efficiency. The inventors have recognized and appreciated that to efficiently illuminate the fundus for imaging and/or measuring, and to avoid the bright reflections caused by the iris, the illumination light should be transmitted such that an outer diameter of the illumination beam has a shorter diameter than a diameter of the pupil of the eye.

The inventors have further recognized that light scattered from front portions of the eye to the detector will decrease the contrast of images and/or measurements acquired of a subject's retina fundus. As discussed above, polarization optics may be used to reduce the transmission of light scattered by front portions of the eye however, polarization optics reduce the intensity of light and therefore result in a decreased energy efficiency because a portion of the generated light that would otherwise be used for imaging is filtered out by the polarization optics. The inventors have appreciated that due to the reflectivity and curvature of the eye, light transmitted to the center of the eye along the optical axis (i.e., perpendicular to the surface of the eye) may result in more scattered light being transmitted to the detector than light that enters the eye at an angle. Accordingly, the inventors have developed an apparatus to illuminate the eye using an illumination profile that is annular (i.e., a ring-shaped) when transmitting through the pupil.

FIG. 15A illustrates an illumination profile 506 generated by the imaging and/or measuring device at a distance corresponding to the subject's pupil during imaging and/or measuring, in accordance with some embodiments. Plot 506 illustrates an annular illumination profile that includes an outer diameter 514 and an inner diameter 515. In some embodiments, the outer diameter is less than or equal to 4.5 mm, less than or equal to 3.8 mm, less than or equal to 3.5 mm, less than or equal to 3 mm and the inner diameter is greater than or equal to 0.5, greater than or equal to 1.5, greater than or equal to 2.5 mm. For example, the illumination profile generated by the imaging and/or measuring device at a distance corresponding to the subject's pupil during imaging and/or measuring may include an outer diameter of 3.5 mm and an inner diameter of 2.5 mm. Other diameters may be used for the outer or inner diameter and other combinations of diameters may be used, as aspects of the technology described herein is not limited in this respect.

FIG. 15B illustrates an illumination profile 508 generated by the imaging and/or measuring device at a distance corresponding to the subject's pupil during imaging and/or measuring, in accordance with some embodiments. Plot 508 illustrates an annular illumination profile that includes an outer diameter 514 and an inner diameter 515. For example, the illumination profile generated by the imaging and/or measuring device may include an outer diameter of 3.5 mm and an inner diameter of 2.5 mm. Other diameters may be used for the inner and outer diameter, as described above in connection with FIG. 15A.

The illumination profile illustrated in plot 508 contains a smaller portion of the illumination light between the inner diameter 515 and the outer diameter 514 than the illumination profile in plot 506. In some embodiments, greater than 40% of the optical power transmitted to the eye from the objective is located between inner diameter 515 and outer diameter 514 at the subject's retina. In other embodiments, greater than 60% of the optical power transmitted to the eye from the objective is located between the inner diameter 515 and outer diameter 514 at the subject's retina. In yet other embodiments, greater than 90% of the optical power transmitted to the eye from the objective is located between the inner diameter 515 and outer diameter 514 at the subject's retina.

In some embodiments, the illumination profile may be approximately symmetric, (e.g., radially and/or axially symmetric). FIG. 15C is a plot illustrating exemplary illumination profiles 520 and 522 generated by the imaging and/or measuring device at a subject's pupil, according to some embodiments. FIG. 15C illustrates line profiles of an illumination profile along a horizontal axis 522 (e.g., a x-axis) and a vertical axis 520 (e.g., a y-axis). As illustrated in FIG. 15C, the profile along the horizontal axis 522 is approximately axially symmetric between the profile at positive coordinates and the profile at negative coordinates. Similarly, the profile along the vertical axis 520 is approximately axially symmetric between the profile at positive coordinates and the profile at negative coordinates. Furthermore, the profile along the horizontal axis 522 is approximately radially symmetric with the profile along the vertical axis 520.

In other embodiments, the illumination profile may include portions of greater intensity and portions of lesser intensity without axial or radial symmetry. In yet other embodiments, the illumination profile may be asymmetric.

FIG. 15D is a plot illustrating exemplary illumination profiles 524 and 526 generated by the imaging and/or measuring device at a subject's pupil, according to some embodiments. FIG. 15D illustrates line profile of an illumination profile along a horizontal axis 524 (e.g., a x-axis) and a vertical axis 526 (e.g., a y-axis). As illustrated in FIG. 15D, the profile along the horizontal axis 524 is not as symmetric as the profiles illustrated in FIG. 15C.

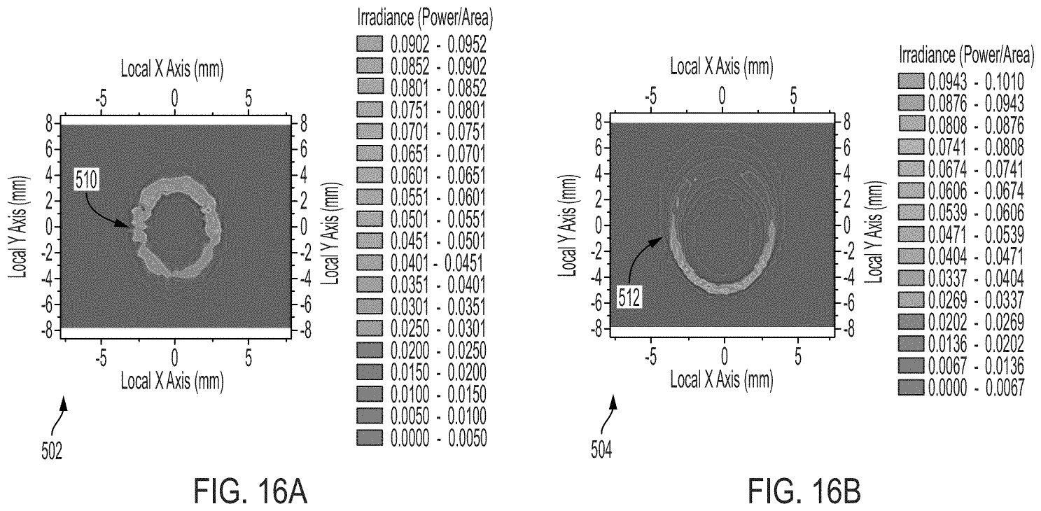

FIG. 16A is an exemplary illumination profile 502 generated by the imaging and/or measuring device at the holed mirror in FIG. 5A, according to some embodiments. The illumination profile illustrated in plot 502 has an annular profile 510. In some embodiments, annular profile 510 is generated by illumination optical components configured to generate an illumination profile to be used with a field of view between 25-35 degrees.

FIG. 16B is another exemplary illumination profile 504 generated by the imaging and/or measuring device at the holed mirror in FIG. 5A, according to some embodiments. The illumination profile illustrated in plot 504 has an annular profile 512. In some embodiments, annular profile 512 is generated by illumination optical components configured to generate an illumination profile to be used with a field of view between 40-50 degrees.

In some embodiments, other illumination profiles may be transmitted to the holed mirror, as aspects of technology described herein are not limited in this respect.

The inventors have appreciated that, in some embodiments, it is advantageous to illuminate the retina fundus with a flat field illumination profile (i.e., an illumination profile that has relatively constant intensity across the field of view). The use of flat field illumination profiles may increase the contrast and/or resolution of features with a detected image and/or measurement. The inventors have further appreciated that an illumination profile that has an annular profile at front portions of the eye and a flat field profile at the back portion of the eye (i.e., at the retina fundus) provides advantages in resolution and/or the contrast of images and/or measurements produced using the illumination light by reducing unwanted reflections and reducing illumination artifacts (e.g., patterns caused by the illumination profile on the retina fundus that may obscure features of the fundus itself) efficiently transmitting light to the relevant portions of the eye.

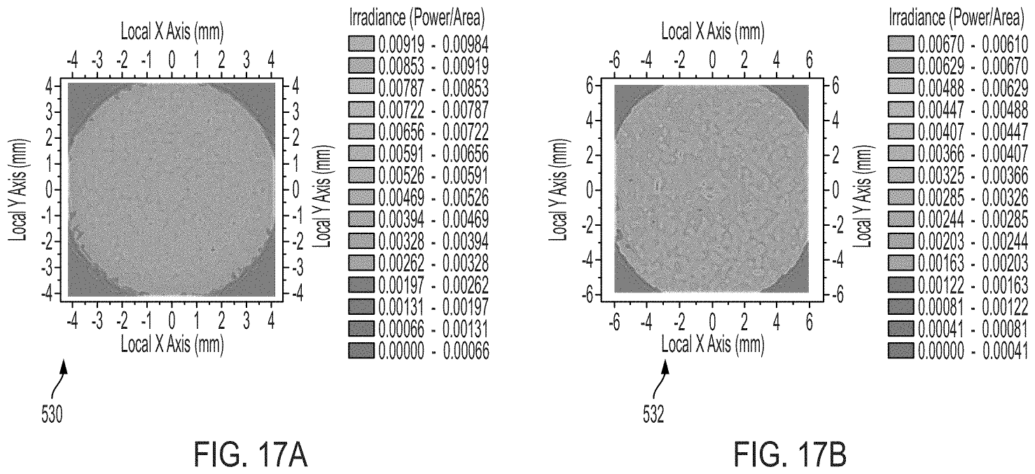

FIG. 17A is an exemplary illumination profile 530 generated by the imaging and/or measuring device at a subject's retina, according to some embodiments. The illumination profile illustrated in plot 530 has a flat field profile. In some embodiments, the illumination profile illustrated in plot 530 is generated by illumination optical components in combination with an objective lens to generate an illumination profile to be used with a field of view between 25-35 degrees.

FIG. 17B is an exemplary illumination profile 532 generated by the imaging and/or measuring device at a subject's retina, according to some embodiments. The illumination profile illustrated in plot 543 has a flat field profile. In some embodiments, the illumination profile illustrated in plot 532 is generated by illumination optical components in combination with an objective lens to generate an illumination profile to be used with a field of view between 40-50 degrees.

VII. Methods for Illuminating and Imaging and/or Measuring a Subject's Eye

In accordance with the aspects of the technology described above, the inventors have developed methods for illuminating and detecting images and/or measurements that may incorporate wide angle fields of view, spatial filtering, and/or interchangeable objective lenses as described herein.

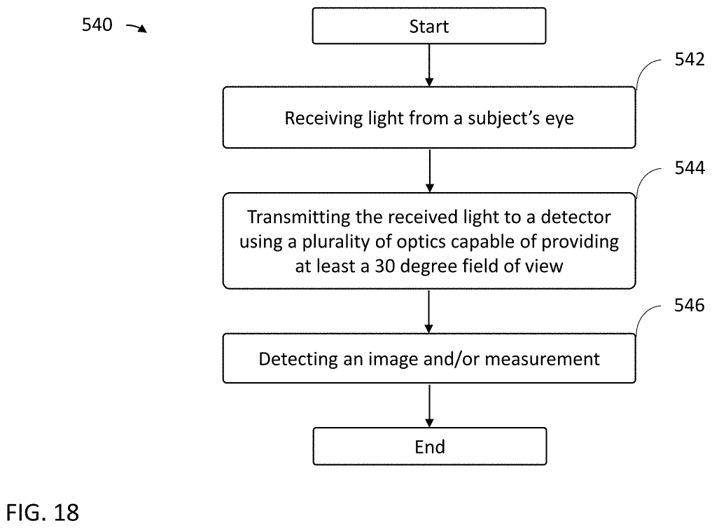

FIG. 18 is a flowchart of a method 540 of detecting an image and/or measurement of a subject's eye, according to some embodiments. In some embodiments, method 540 may be performed using any of the apparatuses described herein and one or more operators (e.g., a user which may include the subject, a technician, nurse, clinician, or any other suitable operator) of the imaging and/or measuring apparatus. Prior to the start of method 540, the operator(s) may select a mode of operation to illuminate a subject's eye, align a subject's eye within a field of view, and or perform diopter compensation.

Method 540 starts at block 542, receiving light from a subject's eye may include an operator(s) aligning the apparatus with a subject's face. For example, the operator(s) may align the apparatus such that light that is reflected and/or emitted from the subject's retina fundus is received by an objective lens. The objective lens receives light, that is within the a of the lens, and transmits the light received by the objective lens to other components of the optical path, as described herein.

In some embodiments, the angular aperture corresponds to a field of view of the subject's retina fundus between 25 and 35 degrees. In some embodiments, the angular aperture corresponds to a field of view of the subject's retina fundus between 40 and 50 degrees. In some embodiments, the angular aperture is much larger than the field of view of the subject's retina fundus. For example, the angular aperture may correspond to a field of view of the subjects retina fundus between 50 and 60 degrees and another optical component may decrease the field of view, as described herein.

Next at block 544, transmitting the received light to a detector using optical components capable of providing at least a 30 degree field of view may occur when the subject's eye is properly aligned with the apparatus. For example, the operator(s) may align the apparatus with the subject's eyes such that the light received by the objective lens is transmitted to the sample and detect components. In some embodiments, the operator(s) may align the apparatus to the subject's eyes prior to receiving light from the subject's eye, such that the light received by the objective lens is transmitted to the sample and detect components. In some embodiments, the operator(s) may align or realign the apparatus to the subject's eye after the apparatus receives light from the eye and prior to detecting the image and/or measurement.

In some embodiments, the holed mirror may be configured to block a portion of the light received from the subject's eye from being transmitted to the detector, as described herein.

Next at block 546, detecting the received light as an image and/or measurement of the subject's retina fundus may include the operator(s) sending a command to detect the received light. For example, the operator(s) may send a command to detect the light received at the detector. The operator(s) may send a command to detect the light using an interface included with the housing or through another device in communication with the imaging and/or measuring apparatus. In some embodiments, the command to detect the light received at the detector may be included in a sequence associated with a selected imaging and/or measuring mode. In some embodiments, the image and/or measurement has a field of view between 25 and 35 degrees. In some embodiments, the image and/or measurement has a field of view between 40 and 50 degrees. In yet other embodiments, the image and/or measurement may have a field of view greater than 50 degrees, as described herein. In some embodiments, a white light image, fluorescence image, optical coherence tomography image, infrared image, and/or other image or measurement is detected, as described herein.

Following the detection of an image and/or measurement, the resulting image and/or measurement may be analyzed in connection with diagnosing a condition of the subject. Additionally, the image may be analyzed to verify that the acquired image meets desired detection parameters such as resolution, contrast, alignment, exposure, and/or focus. If the image and/or measurement is determined not to have met the desired detection parameters, the method may repeat method 540. In some embodiments, method 540 may be repeated after changing parameters of the light source to perform a different imaging and/or measuring process.

As discussed above, the inventors have appreciated that illuminating the retina fundus portion of a subject's eye provides challenges. Light scattered by the front portions of the subject's eye (e.g., the cornea, lens, or iris) and/or light scattered by surfaces of the optical components in the optical path may decrease contrast at the detector. The inventors have developed methods based on the imaging and/or measuring apparatus described herein to overcome these challenges, in accordance with some embodiments.

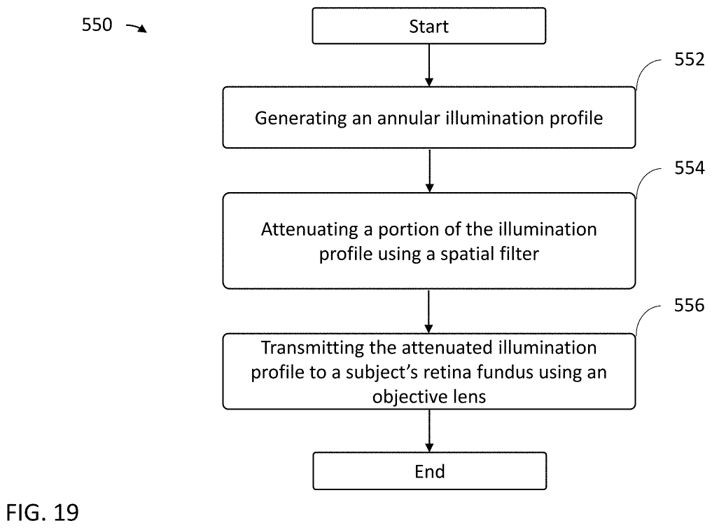

FIG. 19 is a flowchart of a method 550 of illuminating a subject's eye, according to some embodiments. In some embodiments, method 550 may be performed using any of the apparatuses described herein and one or more operators (e.g., a user which may include the subject, a technician, nurse, clinician, or any other suitable operator) of the imaging and/or measuring apparatus. Prior to the start of method 550, the operator(s) may select a mode of operation to specify the wavelengths to be emitted from the light source. For example, for a light source that includes several different light emitters, for emitting different wavelengths, a white light illumination mode may be selected. In some embodiments an infrared imaging mode, or a fluorescence imaging mode may be selected. In some embodiments, fixation light may first be transmitted to the subject's eye to indicate a position of the subject's eye relative to a field of view of the apparatus, as described herein.

Method 550 starts at block 552, generating an annular illumination profile may include light generating components receiving a signal causing them to generate light in response to an operator(s) command. The light generating components (e.g., LEDs) may be arranged in an annular configuration, as shown in FIGS. 8A and 8B and described above, such that when the LEDs receive a signal, causing the LEDs to generate an intensity of light, the light is generated according to an annular illumination profile. For example, the operator(s) may send a command to initiate method 550 either alone or in combination with an imaging and/or measuring command. In some embodiments, the command may involve an interface included with the housing of the apparatus. Other inputs may also be used, as aspects of the technology described herein are not limited in this respect.

In some embodiments, a plate with an annulus is used to block portions of the generated light to modify the illumination profile. The plate with the annulus may modify an annular or non-annular illumination profile generated by the light generating components to produce an illumination profile with an initial inner and outer radius. The plate with the annulus may be plate 362/462 as described above.

Next at block 554, attenuating a portion of the illumination profile may include using a spatial filter to block or obscure a portion of the generated illumination light. In some embodiments, a plate including an obscuration, such as plate 364/464, may be used as the spatial filter. For example, the light generated when the operator(s) initiate method 550 illuminates the spatial filter blocking and/or attenuating a portion of the generated light. The spatial filter may be positioned at approximately a conjugate focal plane to a surface of the objective lens to block and/or attenuate a portion of illumination light from being transmitted to the objective lens. In some embodiments, the positioning of the spatial filter may modify the illumination profile on a surface of the objective, as described above.

In some embodiments, the positioning of the spatial filter may be configured to block and/or attenuate light transmitted at specific angles from being transmitted to the objective lens. The spatial filter may be configured to obscure light rays propagating at specific angles that could reflect off a surface of the objective lens towards the detection components, as described above.

In some embodiments, multiple spatial filters may be included and configured at conjugate focal planes for different surfaces of the objective lens. The spatial filters may be sized to account for a magnification of the spatial filter by other lenses in the optical path. Additionally, the spatial filters may have any suitable shape, as described herein.

Next at block 556, transmitting the attenuated illumination profile may include using an objective lens to transmit the attenuated illumination profile to a subject's retina fundus. For example, the light generated when the operator(s) initiate method 550 illuminates optical components, that may include the spatial filter and the objective lens, to transmit the light to the subject's eye forming an illumination profile on portions of the subject's eye. In some embodiments, the operator(s) may align the apparatus with the subject's face such that light is transmitted from the apparatus to the subject's eye. Collecting lenses 314 and relay lenes 318 (see FIGS. 5A and 5B and description above) may magnify or shrink the annular illumination profile, changing the inner and outer diameter, when transmitting the illumination light to the holed mirror, such as mirror with aperture 322 described above. Exemplary illumination profiles on the holed mirror are illustrated in FIGS. 16A and 16B above. Additionally, the annular illumination profile may be additionally magnified or shrunk by the objective lens and/or the cornea of the subject's eye when transmitting the illumination profile to the subject's eye, such that an illumination profile at subject's pupil has an illumination profile with an inner radius and an outer radius. Exemplary illumination profiles are illustrated in FIGS. 15A-15D above, in accordance with some embodiments.

In some embodiments, the inner diameter of the illumination profile at the pupil of the subject's eye is greater than or equal to 1.8 mm, 2.0 mm, 2.5 mm, or 2.8 mm. In some embodiments the outer diameter of the illumination profile at the pupil of the subject's eye is less than or equal to 4.0 mm, 3.8 mm, 3.5 mm or 3.2 mm. In some embodiments, at least 40% of the optical power received by the retina fundus is localized between the inner and outer diameter of the illumination profile at the pupil of the subject's eye, as described herein.

The illumination light at a subject's retina fundus may have a flat field illumination profile, in accordance with some embodiments. A flat field illumination profile may increase the resolution and/or contrast of the images and/or measurements acquired using light reflected from the illumination profile on the retina fundus, as described above.

After the illumination profile is transmitted to the subject's retina fundus, method 550 ends. Following method 550, a method of detecting or measuring light, such as method 540, may begin, in accordance with some embodiments. In other embodiments, the light transmitted to the subject's eye may be used to determine an alignment of the subject's eye within a field of view of the apparatus. In some embodiments, method 550 may be repeated during alignment of the subject's eye with the field of view.

Having thus described several aspects and embodiments of the technology set forth in the disclosure, it is to be appreciated that various alterations, modifications, and improvements will readily occur to those skilled in the art. Such alterations, modifications, and improvements are intended to be within the spirit and scope of the technology described herein. For example, those of ordinary skill in the art will readily envision a variety of other means and/or structures for performing the function and/or obtaining the results and/or one or more of the advantages described herein, and each of such variations and/or modifications is deemed to be within the scope of the embodiments described herein. Those skilled in the art will recognize, or be able to ascertain using no more than routine experimentation, many equivalents to the specific embodiments described herein. It is, therefore, to be understood that the foregoing embodiments are presented by way of example only and that, within the scope of the appended claims and equivalents thereto, inventive embodiments may be practiced otherwise than as specifically described. In addition, any combination of two or more features, systems, articles, materials, kits, and/or methods described herein, if such features, systems, articles, materials, kits, and/or methods are not mutually inconsistent, is included within the scope of the present disclosure.

The above-described embodiments can be implemented in any of numerous ways. One or more aspects and embodiments of the present disclosure involving the performance of processes or methods may utilize program instructions executable by a device (e.g., a computer, a processor, or other device) to perform, or control performance of, the processes or methods. In this respect, various inventive concepts may be embodied as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other tangible computer storage medium) encoded with one or more programs that, when executed on one or more computers or other processors, perform methods that implement one or more of the various embodiments described above. The computer readable medium or media can be transportable, such that the program or programs stored thereon can be loaded onto one or more different computers or other processors to implement various ones of the aspects described above. In some embodiments, computer readable media may be non-transitory media.

The terms “program” or “software” are used herein in a generic sense to refer to any type of computer code or set of computer-executable instructions that can be employed to program a computer or other processor to implement various aspects as described above. Additionally, it should be appreciated that according to one aspect, one or more computer programs that when executed perform methods of the present disclosure need not reside on a single computer or processor, but may be distributed in a modular fashion among a number of different computers or processors to implement various aspects of the present disclosure.

Computer-executable instructions may be in many forms, such as program modules, executed by one or more computers or other devices. Generally, program modules include routines, programs, objects, components, data structures, etc. that perform particular tasks or implement particular abstract data types. Typically the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that convey relationship between the fields. However, any suitable mechanism may be used to establish a relationship between information in fields of a data structure, including through the use of pointers, tags or other mechanisms that establish relationship between data elements.

When implemented in software, the software code can be executed on any suitable processor or collection of processors, whether provided in a single computer or distributed among multiple computers.

Further, it should be appreciated that a computer may be embodied in any of a number of forms, such as a rack-mounted computer, a desktop computer, a laptop computer, or a tablet computer, as non-limiting examples. Additionally, a computer may be embedded in a device not generally regarded as a computer but with suitable processing capabilities, including a Personal Digital Assistant (PDA), a smartphone or any other suitable portable or fixed electronic device.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible formats.

Such computers may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks, wired networks or fiber optic networks.

The acts performed as part of the methods may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The terms “front” and “rear,” used herein in the context of describing the exemplary imaging and/or measuring apparatuses and portions thereof shown in the drawings, refer to portions of the imaging and/or measuring apparatus facing and/or positioned proximate the subject to be imaged and facing and/or positioned opposite from the subject to be imaged, respectively. It should be appreciated that imaging and/or measuring apparatuses could take other forms in which elements or views described herein as “front” or “rear” may other directions or be positioned differently with respect to the subject or subjects to be imaged, as embodiments described herein are not so limited.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

What is claimed is:

1. An imaging and/or measuring apparatus comprising a lens at least capable of providing a 30 degree field of view of a subject's eye.

2. The imaging and/or measuring apparatus of claim 1, wherein the lens is an objective lens and the imaging and/or measuring apparatus further comprises:
a plurality of illumination optical components configured to transmit light to the objective lens to illuminate a portion of the subject's eye; and
a plurality of fixation optical components configured to transmit light to the objective lens to provide a visual indicator to the subject's eye.

3. The imaging and/or measuring apparatus of claim 2, wherein the objective lens is configured to transmit light received from the subject's eye through an adjustable lens to a detector.

4. The imaging and/or measuring apparatus of claim 3, further comprising a holed mirror, the holed mirror comprising:
a reflective surface configured to transmit light to the objective lens; and
a hole disposed in the reflective surface of the holed mirror, the hole configured to receive light from the objective lens.

5. The imaging and/or measuring apparatus of claim 4, wherein the reflective surface is configured to transmit light received from the plurality of illumination optical components, and wherein the hole is configured to transmit the light received from the objective to the detector.

6. The imaging and/or measuring apparatus of claim 2, wherein the fixation optical path comprises an adjustable lens configured to receive indicator light from a display and to transmit the received indicator light to the objective lens.

7. The imaging and/or measuring apparatus of claim 1, wherein the lens and the plurality of illumination optical components are configured to illuminate a 45 degree field of view of a subject's retina fundus.

8. An imaging and/or measuring apparatus comprising a plurality of imaging and/or measuring optical components configured to:
when a first objective lens is positioned between the plurality of imaging and/or measuring optical components and a subject's eye, transmit and/or receive light having a first set of optical characteristics; and
when a second objective lens is positioned between the plurality of imaging and/or measuring optical components and the subject's eye, transmit and/or receive light a second set of optical characteristics.

9. The imaging and/or measuring apparatus of claim 8, wherein the first set of optical characteristic comprises a first field of view, and the second set of optical characteristics comprises a second field of view.

10. The imaging and/or measuring apparatus of claim 8, wherein the plurality of imaging and/or measuring optical components is configured to provide a field of view between 40 and 60 degrees when configured with the first objective lens.

11. The imaging and/or measuring apparatus of claim 8, wherein the plurality of imaging and/or measuring optical components is configured to provide a field of view between 25 and 35 degrees when configured with the second objective lens.

12. The imaging and/or measuring apparatus of claim 8, further comprising:
a plurality of illumination optical components configured to transmit light to the plurality of imaging and/or measuring optical components to illuminate a portion of the subject's eye; and
a plurality of fixation optical components configured to transmit light to the plurality of imaging and/or measuring optical components to provide a visual indicator to the subject.

13. The imaging and/or measuring apparatus of claim 12, wherein the imaging and/or measuring apparatus further comprises:
a first adjustable lens comprising a plurality of optical components, the first adjustable lens configured to transmit light received from the objective lens to a detector; and
a motor configured to adjust at least one optical component of the first adjustable lens.

14. The imaging and/or measuring apparatus of claim 12, wherein the plurality of fixation optical components comprises a second adjustable lens, the second adjustable lens comprising a second plurality of optical components, wherein the motor is configured to adjust at least one optical component of the second adjustable lens.

15. The imaging and/or measuring apparatus of claim 12, wherein the plurality of illumination optical components is configured to illuminate a 40-60 degree field of view of the subject's eye when configured with the first objective lens.

16. The imaging and/or measuring apparatus of claim 8, wherein the plurality of illumination optical components comprises at least one adjustable optical component.

17. A method of imaging and/or measuring a subject's eye, the method comprising:
receiving light from the subject's eye;
transmitting the received light to a detector using a plurality of imaging and/or measuring optical components, the plurality of imaging and/or measuring optical components at least capable of providing a 30 degree field of view; and
detecting an image and/or measurement.

18. The method of claim 17, further comprising transmitting illumination light to the subject's eye to illuminate a portion of the subject's eye comprising the field of view.

19. The method of claim 18, wherein transmitting illumination light to the subject's eye comprises illuminating a portion of the subject's retina fundus of the subject's eye with a flat field illumination profile.

20. The method of claim 18, wherein transmitting illumination light to the subject's eye comprises illuminating a portion of the subject's retina fundus with unpolarized light.

21. The method of claim 18, further comprising:
receiving light at the objective lens from the fixation path, wherein the light is indicative of an alignment of the subject in the field of view; and
transmitting the light received from the fixation path to the to the subject's eye.
